(19) United States
(12) Patent Application Publication
(10) Pub. No.: US 2025/0100975 A1
(43) Pub. Date: Mar. 27, 2025

(54) TRYPTAMINE ANALOGUES

(71) Applicant: Beckley Psytech Limited, Oxford (GB)

(72) Inventors: Massimo MURATORE, Oxford (GB); Amir Lotfi MOGHADDAM, Oxford (GB); Vinod VISHWAPATHI, Oxford (GB); Christopher WONG, Oxford (GB); Oliver MCGAW, Oxford (GB)

(21) Appl. No.: 18/272,439

(22) PCT Filed: Jan. 17, 2022
(86) PCT No.: PCT/IB2022/050357

§ 371 Date: Jul. 14, 2023

(30) Foreign Application Priority Data
Jan. 15, 2021 (GB) 2100562.4
May 18, 2021 (GB) 2107107.1

(51) Int. Cl. C07D 233/70; A61K 31/4045; A61K 31/4184; A61K 31/4439; A61K 31/5386; C07D 209/16; C07D 213/643; C07D 309/12; C07D 498/08

(52) U.S. Cl. CPC C07D 233/70; A61K 31/4045; A61K 31/4184; A61K 31/4439; A61K 31/5386; C07D 209/16; C07D 213/643; C07D 309/12; C07D 498/08

(57) ABSTRACT

This invention relates to pharmaceutically acceptable tryptamine analogues and salts thereof. In particular, though not exclusively, the a invention relates to formulations and uses of the same as a medicament.

### FIELD OF THE INVENTION

This invention relates to pharmaceutically acceptable tryptamine analogues and salts thereof. In particular, though not exclusively, the invention relates to formulations and uses of the same as a medicament.

### BACKGROUND TO THE INVENTION

Tryptamine is an indolamine metabolite of the essential amino acid, tryptophan. The chemical structure is defined by an indole—a fused benzene and pyrrole ring, and a 2-aminoethyl group at the third carbon. The structure of tryptamine is:

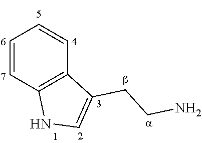 [SMILES: NCCc1c[nH]c2ccccc12]

The structure of tryptamine is a shared feature of certain aminergic neuromodulators including melatonin, serotonin, bufotenin as well as psychedelic derivatives such as dimethyltryptamine (DMT), psilocybin, psilocin.

Various synthetic modifications to the structure of tryptamine have been made, e.g. structural changes at the 1, 4, 5, 6 and 7 positions. However, such modifications often result in a decrease in activity. Ineffective docking/binding of these compounds to the appropriate receptors may result from such structural modifications.

There remains a need in the art for tryptamine-based therapeutics.

### SUMMARY

Herein disclosed a compound of Formula (1)

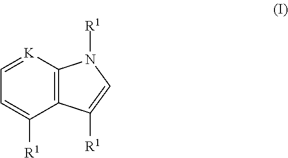

wherein:
R¹ is selected from H or C₁₋₆ alkyl; and
R² is selected from H, NH₂-cyclopropyl or CH₂—CHR⁴—NR⁵R⁶, wherein
R⁴ is selected from H or C₁₋₆ alkyl, wherein
R⁵ is selected from H or C₁₋₆ alkyl, wherein
R⁶ is selected from C₁₋₆ alkyl, A—(CH₂)ₙ—B or

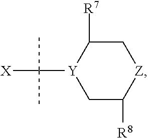

wherein
A is NH or O, wherein
B is NH₂, NH(C₁₋₆ alkyl), N(C₁₋₆ alkyl)₂ or OH, wherein
n is 0 to 4, wherein
X is a bond, NH or O, wherein
Y is selected from CH, N, wherein
Z is selected from CH₂, O, NH or N(C₁₋₆ alkyl), wherein
R⁷ and R⁸ are H, or R⁷ and R⁸ together from a bridging group CH₂CH₂, CH₂O or CH₂NH, wherein
R⁵ and R⁶ are different; and
R³ is selected from H or L—R⁹, wherein
L is selected from O, N, NH, O—O, CH₂, CH₂CH₂, O—O—CH₂CH₂, CO, OCO, COO or OCONH, wherein
R⁹ is selected from H, C₁₋₆ alkyl, COOH, OH, COO—, O—, C₆-C₁₀ aromatic, C₃-C₁₀ hetroaromatic or heterocyclic group comprising one, two or three heteroatoms independently selected from O and N; and
K is CH, or the group defined by K═CH—CH═CR³ is NH—CH═CH—C(O);
or is a pharmaceutically acceptable salt thereof.

Herein disclosed R⁵ and R⁶ are C₁₋₆ alkyl groups.

Herein disclosed R⁶ is methyl.

Herein disclosed R⁶ is

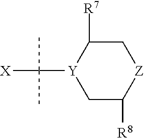

and wherein X is a bond, Y is N, Z is O and R⁷ and R⁸ together from a bridging group CH₂CH₂.

Herein disclosed R⁶ is

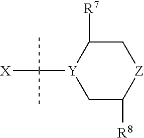

wherein X is NH or O, Y is CH, Z is O and R⁷ and R⁸ are H.

Herein disclosed R⁶ is NH(CH₂)₃N(CH₃)₂, O(CH₂)OH or O(CH₂)₂N(CH₃)₂.

In a first aspect of the invention, there is provided a compound of Formula (1) a compound of Formula (1) wherein:

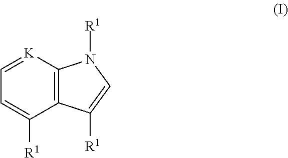

R¹ is selected from H or C₁₋₆ alkyl; and
R² is CH₂—CHR⁴—NR⁵R⁶, wherein
   R⁴ is selected from H or C₁₋₆ alkyl, wherein
   R⁵ is selected from H or C₁₋₆ alkyl, wherein
   R⁶ is:
a)

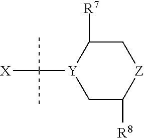

wherein X is a bond, Y is N, Z is O and R⁷ and R⁸ together from a bringing group CH₂CH₂; or
b)

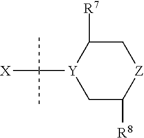

wherein X is NH or O, Y is CH, Z is O and R⁷ and R⁸ are H; or
c) NH(CH₂)₃N(CH₃)₂, O(CH₂)OH or O(CH₂)₂N(CH₃)₂.
R³ is selected from H or L—R⁹, wherein
   L is selected from O, N, NH, O—O, CH₂, CH₂CH₂, O—O—CH₂CH₂, CO, OCO, COO or OCONH, wherein
   R⁹ is selected from H, C₁₋₆ alkyl, COOH, OH, COO—, O—, C₆-C₁₀ aromatic, C₃-C₁₀ heteroaromatic or heterocyclic group comprising one, two or three heteroatoms independently selected from O and N; and
K is CH, CH—CH═CR³ or NH—CH═CH—C(O);
or is a pharmaceutically acceptable salt thereof.

In an embodiment the alkyl group is straight, branched or a cyclic alkyl group.

In an embodiment the alkyl group is a straight chain alkyl group. In an embodiment the alkyl group contains 1, 2 or 3 halogens.

In an embodiment R¹ is H, R² is CH₂—CHR⁴—NR⁵R⁶, R³ is H or L—R⁹, and K is CH.

In an embodiment R⁴ is H or methyl.

In an embodiment R⁵ is ethyl or isopropyl.

In an embodiment R⁴ is H and R⁵ is ethyl.
In an embodiment R³ is L—R⁹ and wherein L is O, —O—CH₂CH₂, OCO or OCONH.

In an embodiment R³ is L—R⁹ and wherein R⁹ is OH, COCH₃, imidazole, pyrrolidine, piperidine, analine, pyridine, morpholine or 6-oxa-3-azabicyclo[3.1.1](heptane.

In an embodiment R³ is OCOCH₃.

In an embodiment there is provided one or more compounds selected from:

| Number | IUPAC | SMILES | Structure |
|---|---|---|---|
| 001 | (S)-ethyl({2-[4-(1H- imidazol-5-yloxy)- 1H-indol-3-yl]ethyl})- methylazanium | [H]N1C([H])═NC([H])═C1OC1═ C2C(N([H])C([H])═C2C([H])([H]) C([H])([H])[N@@+]([H])(C([H]) ([H])[H])C([H])([H])C([H])([H]) [H])═C([H])C([H])═C1[H] | 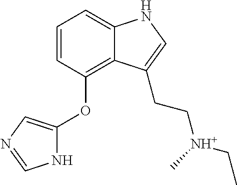 [SMILES: CC[N@H+](C)CCc1c[nH]c2cccc(Oc3cnc[nH]3)c12] |
| 002 | (R)-ethyl(methyl)[2- (4-{[2-(pyrrolidin-1- yl)ethyl]peroxy}-1H- indol-3-yl)ethyl]- azanium | [H]N1C([H])═C(C2═C(OOC([H]) ([H])C([H])([H])N3C([H])([H])C ([H])([H])C([H])([H])C3([H])([H]) C([H])═C([H])C([H])═C12)C([H]) ([H])C([H])([H])[N@+]([H])(C ([H])([H])([H])C([H])([H])C([H]) ([H])[H] | 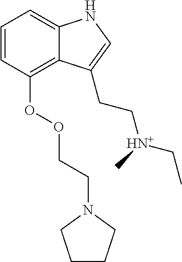 [SMILES: CC[N@@H+](C)CCc1c[nH]c2cccc(OOCCN3CCCC3)c12] |
| 003 | (R)-ethyl(methyl)(2- {4-[(15,4S)-2-oxa-5- azabicyclo[2.2.1] heptan-5-yloxy]-1H- indol-3-yl}ethyl)- azanium | [H]N1C([H])═C(C2═C(ON3C([H]) ([H])[C@@]4([H])OC([H])([H]) [C@]3([H])C4([H])[H])C([H])═ C([H])C([H])═C12)C([H])([H])C ([H])([H])[N@+]([H])(C([H])([H]) [H])C([H])([H])C([H])([H])[H] | 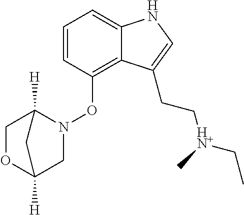 |
| 004 | (S)-ethyl(methyl)(2- {4-[(1R,5S)-6-oxa-3- azabicyclo[3.1.1] heptan-3-yloxy]-1H- indol-3-yl}ethyl)- azanium | [H]N1C([H])═C(C2═C(ON3C([H]) ([H])[C@@]4([H])O[C@]([H])(C4 ([H])[H])C3([H])[H])C([H])═C ([H])C([H])═C12)C([H])([H])C ([H])([H])[N@@+]([H])(C([H]) ([H])[H])C([H])([H])C([H])([H]) [H] | 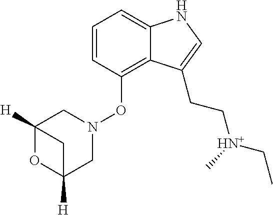 [SMILES: CC[N@H+](C)CCc1c[nH]c2cccc(ON3C[C@H]4C[C@@H](C3)O4)c12] |
| 005 | (S)-ethyl(methyl)(2- {4-[(phenylcarbam- oyl)oxy]-1H-indol-3- yl}ethyl)azanium | [H]N(C(═O)OC1═C2C(N([H])C ([H])═C2C([H])([H])C([H])([H]) [N@@+]([H])(C([H])([H])[H])C ([H])([H])C([H])([H])[H])═C([H]) C([H])═C1[H])C1═C([H])C ([H])═C([H])C([H])═C1[H] | 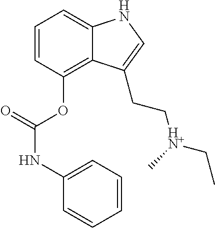 |
| 006 | (R)-ethyl(2-{4-[(2- hydroxyethyl)peroxy]- 1H-indol-3-yl}ethyl)- methylazanium | [H]OC([H])([H])C([H])([H]) OOC1═C2C(N([H])C([H])═C2C ([H])([H])C([H])([H])[N@+]([H]) (C([H])([H])[H])C([H])([H])C ([H])([H])[H])═C([H])C([H])═ C1[H] | 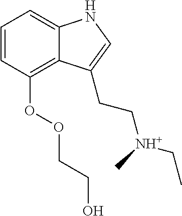 [SMILES: CC[N@@H+](C)CCc1c[nH]c2cccc(OOCCO)c12] |
| 007 | 3-{2-[(R)-ethyl- (methyl)azaniumyl] ethyl}-1H-indol-4-yl carbonate | [H]N1C([H])═C(C2═C(OC ([O—])═O)C([H])═C([H])C ([H])═C12)C([H])([H])C([H]) ([H])[N@+]([H])(C([H])([H]) [H])C([H])([H])C([H])([H])[H] | 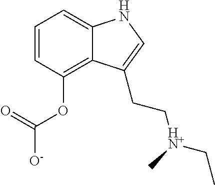 |
| 008 | (S)-ethyl(methyl){2- [4-(pyridin-2-yloxy)- 1H-indol-3-ylethyl}- azanium | [H]N1C([H])═C(C2═C(OC3═C ([H])C([H])═C([H])C([H])═N3) C([H])═C([H])C([H])═C12)C ([H])([H])C([H])([H])[N@@+] ([H])(C([H])([H])[H])C([H])([H]) C([H])([H])[H] | 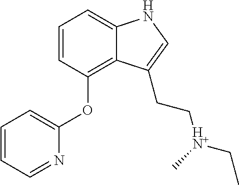 |
| 009 | (S,1R,4R)-N-{2-[4- (acetyloxy)-1H- indol-3-ylethyl}-N- ethyl-2-oxa-5-aza- bicyclo[2.2.2]octan- 5-aminium | [H]N1C([H])═C(C2═C(OC(═O) C([H])([H])[H])C([H])═C([H])C ([H])═C12)C([H])([H])C([H])([H]) [N@@+]([H])(N1C([H])([H])[C@] 2([H])OC([H])([H])[C@@]1([H]) C([H])([H])C2([H])[H])C([H])([H]) C([H])([H])[H] | 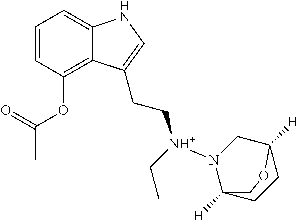 [SMILES: CC[N@@H+](CCc1c[nH]c2cccc(OC(C)=O)c12)N1C[C@H]2CC[C@@H]1CO2] |
| 010 | (1R)-1-{2-[4-(acet- yloxy)-1H-indol-3- yl]ethyl}-1-ethyl-2- (oxan-4-yl)hydra- zinium | [H]N(C1([H])C([H])([H])C([H]) ([H])OC([H])([H])C1([H])[H]) (N@+]([H])(C([H])([H])C([H]) ([H])[H])C([H])([H])C([H])([H]) C1═C([H])N([H])C2═C([H])C ([H])═C([H])C(OC(═O)C([H]) ([H])[H])═C12 | 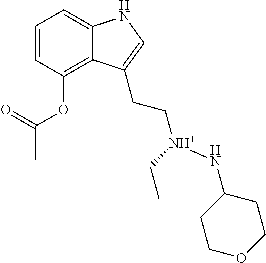 [SMILES: CC[N@H+](CCc1c[nH]c2cccc(OC(C)=O)c12)NC1CCOCC1] |
| 011 | (S)-{2-[4-(acetyl- oxy)-1H-indol-3-yl]- ethyl}(ethyl)(oxan- 4-yloxy)azanium | [H]N1C([H])═C(C2═C(OC(═O) C([H])([H])[H])C([H])═C([H])C ([H])═C12)C([H])([H])C([H]) ([H])[N@@+]([H])(OC1([H])C ([H])([H])C([H])([H])OC([H]) ([H])C1([H])[H])C([H])([H])C ([H])([H])[H] | 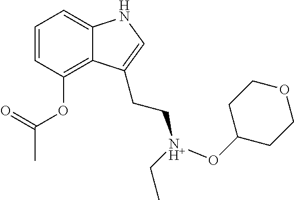 [SMILES: CC[N@@H+](CCc1c[nH]c2cccc(OC(C)=O)c12)OC1CCOCC1] |
| 012 | 2-{2-[4-(acetyloxy)- 1H-indol-3-yl]- ethyl}-1-[3-(dimeth- ylazaniumyl)prop- yl]-2-ethylhydra- zinium | [H]N1C([H])═C(C2═C(OC(═O) C([H])([H])[H])C([H])═C([H]) C([H])═C12)C([H])([H])C([H]) ([H])N(C([H])([H])C([H])([H]) [H])[N+]([H])([H])C([H])([H]) C([H])([H])C([H])([H])[N+]([H]) (C([H])([H])[H])C([H])([H])[H] | 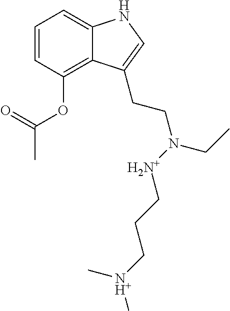 [SMILES: CCN(CCc1c[nH]c2cccc(OC(C)=O)c12)[NH2+]CCC[NH+](C)C] |
| 013 | (R)-{2-[4-(acetyl- oxy)-1H-indol-3-yl]- ethyl}(ethyl)(3- hydroxypropoxy)- azanium | [H]OC([H])([H])C([H])([H])C ([H])([H])O[N@+]([H])(C([H]) ([H])C([H])([H])[H])C([H])([H]) C([H])([H])C1═C([H])N([H]) C2═C([H])C([H])═C([H])C(OC (═O)C([H])([H])[H])═C12 | 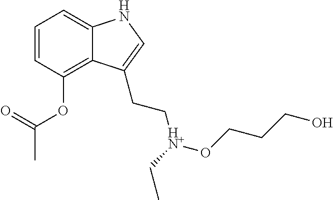 |
| 014 | {2-[{{2-[4-(acetyl- oxy)-1H-indol-3-yl]- ethyl}(ethyl)amino)- oxy]ethyl}dimethyl- azanium | [H]N1C([H])═C(C2═C(OC(═O) C([H])([H])[H])C([H])═C([H]) C([H])═C12)C([H])([H])C([H]) ([H])N(OC([H])([H])C([H])([H]) [N+]([H])C([H])([H])[H])C([H]) ([H])[H])C([H])([H])C([H])([H]) [H] | 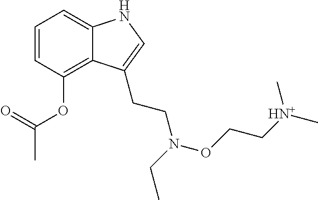 [SMILES: CCN(CCc1c[nH]c2cccc(OC(C)=O)c12)OCC[NH+](C)C] |
| 015 | (S,1R,4R)-N-{2-[4- (acetyloxy)-1H- indol-3-ylethyl}-N- ethyl-2-oxa-5-aza- bicyclo[2.2.2]octan- 5-aminium | [H]N1C([H])═C(C2═C(OC(═O) C([H])([H])[H])C([H])═C([H])C ([H])═C12)C([H])([H])C([H]) ([H])[N@@+]([H])(N1C([H]) ([H])[C@]2([H])OC([H])([H]) [C@@]1([H])C([H])([H])C2 ([H])[H])C([H])([H])C([H])([H]) [H] | 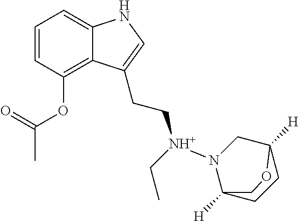 [SMILES: CC[N@@H+](CCc1c[nH]c2cccc(OC(C)=O)c12)N1C[C@H]2CC[C@@H]1CO2] |
| 016 | (S)-N-{2-[4-(acetyl- oxy)-1H-indol-3-yl]- ethyl}-N-ethyl-4- methylpiperazin-1- aminium | [H]N1C([H])═C(C2═C(OC(═O) C([H])([H])[H])C([H])═C([H])C ([H])═C12)C([H])([H])C([H]) ([H])(N@@+]([H])(N1C([H])([H]) C([H])([H])N(C([H])([H])[H])C ([H])([H])C1([H])[H])C([H])([H]) C([H])([H])[H] | 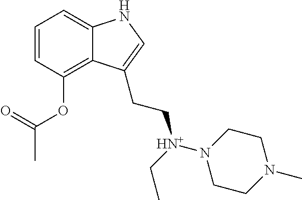 [SMILES: CC[N@@H+](CCc1c[nH]c2cccc(OC(C)=O)c12)N1CCN(C)CC1] |
| 017 | (R)-[2-(1H-indol-3- yl)ethyl](methyl)- propylazanium | [H]N1C([H])═C(C2═C1C ([H])═C([H])C([H])═C2[H])C ([H])([H])C([H])([H])[N@+]([H]) (C([H])([H])[H])C([H])([H])C ([H])([H])C([H])([H])[H] | 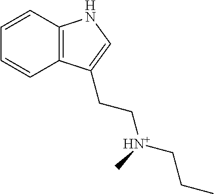 [SMILES: CCC[N@@H+](C)CCc1c[nH]c2ccccc12] |
| 018 | (R)-[(2R)-1-(1H- indol-3-yl)propan-2- yl](methyl)(propan- 2-yl)azanium | [H]N1C([H])═C(C2═C1C ([H])═C([H])C([H])═C2[H])C ([H])([H])[C@@]([H])(C([H]) ([H])[H])[N@+]([H])(C([H]) ([H])[H])C([H])(C([H])([H]) [H])C([H])([H])[H] | 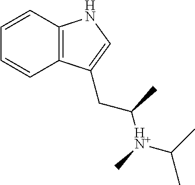 |
| 019 | 1-pentyl-1H,4H,7H- pyrrolo[2,3-b]pyr- idin-4-one | [H]N1C([H])═C([H])C(═O) C2═C1N(C([H])═C2[H])C([H]) ([H])C([H])([H])C([H])([H])C ([H])([H])C([H])([H])[H] | 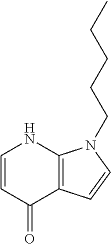 |
| 020 | (1S,2R)-2-(1H-indol- 3-yl)cyclopropan-1- aminium | [H]N1C([H])═C(C2═C1C ([H])═C([H])C([H])═C2[H]) [C@@]1([H])C([H])([H]) [C@]1([H])[N+]([H])([H])[H] | 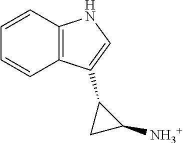 [SMILES: [NH3+][C@H]1C[C@@H]1c1c[nH]c2ccccc12] |

As calculated and described further herein below, compounds 001 to 020 have good ‘Docking Scores’ (Kcal/mol) to target the modelled receptors and are synthetically accessible. As such, compounds 001 to 020 are demonstrated to be synthetically accessible and useful as medicaments for appropriate conditions involving the target receptors, or related receptors with the associated corresponding conditions.

| Number | Synthetic Accessibility | Docking Scores (Kcal/mol) |
|---|---|---|
| 001 | 2.79 | −12.691 |
| 002 | 3.36 | −11.829 |
| 003 | 4.67 | −11.791 |
| 004 | 4.61 | −11.629 |
| 005 | 2.88 | −11.176 |
| 006 | 2.95 | −11.051 |
| 007 | 2.27 | −10.691 |
| 008 | 2.76 | −10.657 |
| 009 | 4.68 | −12.441 |
| 010 | 3.09 | −11.148 |
| 011 | 3.17 | −11.018 |
| 012 | 3.15 | −10.88 |
| 013 | 3 | −10.628 |
| 014 | 3.13 | −10.027 |
| 015 | 4.68 | −9.917 |
| 016 | 3.19 | −9.824 |
| 017 | 1.82 | −10.463 |
| 018 | 2.47 | −9.724 |
| 019 | 1.78 | −5.853 |
| 020 | 2.35 | −8.579 |

In an embodiment there is provided a composition comprising a pharmaceutically effective amount of a compound as described previously.

In an embodiment the composition comprises a dosage amount in the range of 0.05 mg to 100 mg.

In an embodiment the composition comprises a dosage amount in the range of 0.1 mg to 50 mg.

In an embodiment the composition comprises a dosage amount in the range of 0.5 mg to 25 mg.

In an embodiment the composition comprises a dosage amount in the range of 0.5 mg to 10 mg.

In an embodiment the composition comprises a dosage amount in the range of 1 mg to 10 mg.

In an embodiment the composition comprises a dosage amount in the range of 1 mg to 8 mg.

In an embodiment the composition comprises a dosage amount in the range of 3 mg to 15 mg.

In an embodiment the composition comprises a dosage amount in the range of 0.005 mg to 100 mg.

In an embodiment the composition comprises a dosage amount in the range of 0.001 mg to 100 mg.

In an embodiment the composition comprises a dosage amount in the range of 0.0005 mg to 100 mg.

The level of the active agent can be adjusted as required by need for example to suit a certain patient group (e.g. the elderly) or the conditions being treated.

In an embodiment the composition is formulated in a dosage form selected from: oral, transdermal, inhalable, intravenous, rectal dosage, intranasal, intramuscular, or any other parenteral form.

In an embodiment the composition is formulated in a dosage form selected from: oral, transdermal, inhalable, intravenous, or rectal dosage form.

It is advantageous to be able to deliver the active agent in different forms, for example to suit a certain patient group (e.g. the elderly) or the conditions being treated.

In an embodiment the composition is formulated in a dosage form selected from: tablet, capsule, granules, powder, free-flowing powder, inhalable powder, aerosol, nebulised, vaping, buccal, sublingual, sublabial, injectable, or suppository dosage form.

In an embodiment the powder is suitable for administration by inhalation via a medicament dispenser selected from a reservoir dry powder inhaler, a unit-dose dry powder inhaler, a pre-metered multi-dose dry powder inhaler, a nasal inhaler or a pressurized metered dose inhaler.

In an embodiment the powder comprises particles, the particles having a median diameter of less than 2000 μm, 1000 μm, 500 μm, 250 μm, 100 μm, 50 μm, or 1 μm.

In an embodiment the powder comprises particles, the particles having a median diameter of greater than 500 μm, 250 μm, 100 μm, 50 μm, 1 μm or 0.5 μm.

In an embodiment the powder comprises particles, and wherein the powder has a particle size distribution of d10=20-60 μm, and/or d50=80-120 μm, and/or d90=130-300 μm.

The nature of the powder can be adjusted to suit need. For example, if being made for nasal inhalation, then the particles may be adjusted to be much finer than if the powder is going to be formulated into a gelatine capsule, or differently again if it is going to be compacted into a tablet.

In an embodiment the compound is in the form of a salt which is amorphous or crystalline.

In an embodiment the salt is in a polymorphic crystalline form.

In an embodiment the salt is a benzoate, fumarate, citrate, acetate, succinate, halide, fluoride, chloride, bromide, iodide, oxalate, or triflate salt, optionally the salt is the chloride, benzoate or fumarate salt.

In an embodiment the salt is formulated into a composition for mucosal delivery. In an embodiment, the salt is a benzoate salt.

For the salt, the dosage amount is the equivalent amount of the free base delivered when the salt is taken. So 100 mg dosage amount may for example correspond to 117 mg of a hydrochloride salt (i.e. both providing the same molar amount of the active substance). The greater mass of the salt needed is due to the larger formula weight of the hydrogen chloride salt. Similarly, for the deuterated or triturated version of the compounds of the invention (also considered within the scope of the invention), a slight increase in mass can be expected due to the increased formula weight of these isotopic compounds.

Amorphous and crystalline substances often show different chemical/physical properties, e.g. improved rate of dissolution in a solvent, or improved thermal stability. Similarly, different polymorphs may also show different and useful chemical/physical properties.

In an embodiment the composition comprises one or more pharmaceutically acceptable carriers or excipients.

In an embodiment the composition comprises one or more of: mucoadhesive enhancer, penetrating enhancer, cationic polymers, cyclodextrins, Tight Junction Modulators, enzyme inhibitors, surfactants, chelators, and polysaccharides.

In an embodiment the composition comprises one or more of: chitosan, chitosan derivatives (such as N,N,N-trimethyl chitosan (TMC), n-propyl-(QuatPropyl), n-butyl-(QuatButyl) and n-hexyl (QuatHexyl)-N,N-dimethyl chitosan, chitosan chloride), β-cyclodextrin, Clostridium perfringens enterotoxin, Zonula occludens toxin (ZOT), human neutrophil elastase inhibitor (ER143), sodium taurocholate, sodium deoxycholate sodium, sodium lauryl sulphate, glycodeoxycholat, palmitic acid, palmitoleic acid, stearic acid, oleyl acid, oleyl alcohol, capric acid sodium salt, DHA, EPA, dipalmitoyl phophatidyl choline, soybean lecithin, lysophosphatidylcholine, dodecyl maltoside, tetradecyl maltoside, EDTA, lactose, cellulose, and citric acid.

In an embodiment the compound or composition defined herein above for use in a method of treatment of a human or animal subject by therapy.

In an embodiment the method of treatment is a method of treatment of:
conditions caused by dysfunctions of the central nervous system,
conditions caused by dysfunctions of the peripheral nervous system,
conditions benefiting from sleep regulation (such as insomnia),
conditions benefiting from analgesics (such as chronic pain), migraines,
trigeminal autonomic cephalgias (such as short-lasting unilateral neuralgiform headache with conjunctival injection and tearing (SUNCT), and short-lasting neuralgiform headaches with cranial autonomic symptoms (SUNA)),
conditions benefiting from neurogenesis (such as stroke, traumatic brain injury, Parkinson's dementia),
conditions benefiting from anti-inflammatory treatment,
depression,
anxiety,
substance use disorder,
addictive disorder,
gambling disorder,
eating disorders,
obsessive-compulsive disorders, or
body dysmorphic disorders,
optionally the condition is SUNCT and/or SUNA.

Treatment of the above conditions may be beneficially improved by taking the invention.

In an embodiment the method of treatment is a method of treatment of more than one of the above conditions, for example, the method of treatment may be a method of treatment of depression and anxiety.

In an embodiment the composition is administered one or more times a year.

In an embodiment the composition is administered one or more times a month.

In an embodiment the composition is administered one or more times a week.

In an embodiment the composition is administered one or more times a day.

In an embodiment the composition is administered at such a frequency as to avoid tachyphylaxis.

In an embodiment the composition is administered together with a complementary treatment and/or with a further active agent.

In an embodiment the further active agent is a psychedelic compound, optionally a further tryptamine.

In an embodiment the further active agent is a psychedelic compound, optionally an ergoline.

In an embodiment the further active agent is lysergic acid diethylamide (LSD), psilocybin, psilocin or a prodrug thereof.

In an embodiment the complementary treatment is psychotherapy.

In an embodiment, there is provided a composition comprising a pharmaceutically effective amount of a compound as described herein for use in a method of treatment of treatment resistant depression.

In an embodiment, there is provided a nasal inhalation composition comprising a pharmaceutically effective amount of a compound as described herein for use in a method of treatment of treatment resistant depression.

Treatment of the above conditions may be beneficially improved by taking the invention together with some complementary treatments; also these treatments may occur much less regularly than some other treatments that require daily treatments or even multiple treatments a day.

### BRIEF DESCRIPTION OF THE FIGURES

FIG. 1 shows serotonin (1a, 1b, 2a, 2c and 7 receptors) cAMP assay results for compounds 005, 008 and 018.

FIG. 2 shows serotonin (2a, 2b and 2c receptors) inositol phosphate 1 (IP₁) assay results for compounds 005, 008 and 018.

FIG. 3 shows serotonin (2a, 2b and 2c receptors) calcium (Ca²⁺) assay results for compounds 005, 008 and 018.

FIG. 4 shows serotonin (1a, 1b, 2a and 2c receptors) B-arrestin assay results for compounds 005, 008 and 018.

FIG. 5 shows serotonin 2b receptor B-arrestin assay results for compounds 005, 008 and 018.

### DETAILED DESCRIPTION OF THE INVENTION

The crystal structures of the serotonin receptor were retrieved from the Protein Data Bank (www.rcsb.org) [PDB ID: 5TVN and 6WGT for 5-HT2B and 5-HT2A respectively. Both proteins were prepared for docking of candidate ligands. Briefly, hydrogens were added, bond orders were assigned, and loops and side chains were filled. Restrain minimization was performed using Optimized Potentials for Liquid Simulations (OPLS2005) force field until the RMSD reached 0.3 Å from the initial geometry in order to improve steric clashes. Additionally, other possible receptor targets were used and prepared structurally as further indicated below.

### Binding Pocket Analysis

Only two crystallographic structures are available from the Protein Data Bank of the 5HT-2A and 5HT-2B receptors in complex with (8alpha)-N,N-diethyl-6-methyl-9,10-didehydroergoline-8-carboxamide which represents an active isoform of LSD. The binding pockets were analysed to determine the interaction between the receptor residues and the ligands structure. Homologies modelling was applied to determine similarities in several receptor/protein targets. Moreover the binding pocket was analysed by intrinsic dynamic Domains (IDD) methodology to further verify the residues on the receptors that most contribute to the activity of the binding site.

### Ligands

Initial consideration regarding anti-inflammatory properties, vaso-constriction, vaso-dilation and psychedelic effects were considered. In tryptamine the indole system was preserved as a starting scaffold. Analysis of key attachment points was implemented and subsequently R-groups were assigned to the initial scaffold. The R-groups were selected from a library of fragments. The compounds were filtered by Lipinski's rule of five (RO5), rapid elimination of swill (REOS) and pan assay interference compounds (PAINS 1, 2, and 3).

The resulting ligand structures were prepared for docking by identifying stereoisomers with protonation states of pH7±2.

### Docking

In the binding pocket, residues containing hydroxyl and thiol groups were rotated to account for some flexibility of the pocket in the first stage of rigid docking. Subsequently the best compounds were used for flexible docking in order to further simulate a physiological state of the receptors.

### Molecular Dynamics Simulation

Simulations for both receptors were implemented on the basis of the top ligand binding scores. All of the simulations were carried out using the MD Desmond package. Available crystal structures were used. The receptor and ligand complexes were set up in an orthorhombic box using a buffer condition of 10 Å. The orientations of the membranes (if available) were from the Orientation of Protein in Membranes (OPM) database. Ions were neutralized for the system, salt was added at a concentration of 0.15M NaCl, and OPLS 2005 was used for the force field. Long-range electrostatic interactions were calculated with the Ewald method using a cut-off of 9 Å for Van der Waals and Coulomb interactions. The simulation was carried out in an isothermal, isobaric ensemble (NPT) with an initial temperature of 300° K and 1 bar of pressure. The temperature followed the Nose-Hoover method and the pressure was controlled by the Martyna-Tobias-Klein protocol. The simulation was set for 200 ns and trajectories were recorded every 100 ps. The default relaxation protocol for the system was used. Representative structures were extrapolated from the simulation at 0, 100, and 200 ns.

### Chemical and Other Pharmacokinetic Properties

The compounds were analysed computationally regarding suitable chemical characteristic and pharmacokinetic parameters and compared across known agonists of mainly the two serotonin receptors (5HT-2A and 2B). Several algorithms were implemented.

### Identified Tryptamine Analogues

The structures of selected tryptamine analogues are shown in the table above and further described below.

### Properties of Selected Tryptamine Analogues

Various properties of selected tryptamine analogues are detailed in the tables below, followed by an explanation of the properties:

| Number | Formula | MW | Heavy atoms |
|---|---|---|---|
| 001 | C16H21N4O | 285.36 | 21 |
| 002 | C19H3ON3O2 | 332.46 | 24 |
| 003 | C18H26N3O2 | 316.42 | 23 |
| 004 | C18H26N3O2 | 316.42 | 23 |
| 005 | C20H24N3O2 | 338.42 | 25 |
| 006 | C15H23N2O3 | 279.35 | 20 |
| 007 | C14H18N2O3 | 262.3 | 19 |
| 008 | C18H22N3O | 296.39 | 22 |
| 009 | C20H28N3O3 | 358.45 | 26 |
| 010 | C19H28N3O3 | 346.44 | 25 |
| 011 | C19H27N2O4 | 347.43 | 25 |
| 012 | C19H32N4O2 | 348.48 | 25 |
| 013 | C17H25N2O4 | 321.39 | 23 |
| 014 | C18H28N3O3 | 334.43 | 24 |
| 015 | C20H28N3O3 | 358.45 | 26 |
| 016 | C19H29N4O2 | 345.46 | 25 |
| 017 | C14H21N2 | 217.33 | 16 |
| 018 | C15H23N2 | 231.36 | 17 |
| 019 | C12H16N2O | 204.27 | 15 |
| 020 | C11H13N2 | 173.23 | 13 |
| | Aromatic | | Rotatable |
| Number | heavy atoms | Csp3 | bonds |
| 001 | 14 | 0.31 | 6 |
| 002 | 9 | 0.58 | 9 |
| 003 | 9 | 0.56 | 6 |
| 004 | 9 | 0.56 | 6 |
| 005 | 15 | 0.25 | 8 |
| 006 | 9 | 0.47 | 8 |
| 007 | 9 | 0.36 | 6 |
| 008 | 15 | 0.28 | 6 |
| 009 | 9 | 0.55 | 7 |
| 010 | 9 | 0.53 | 8 |
| 011 | 9 | 0.53 | 8 |
| 012 | 9 | 0.53 | 11 |
| 013 | 9 | 0.47 | 10 |
| 014 | 9 | 0.5 | 10 |
| 015 | 9 | 0.55 | 7 |
| 016 | 9 | 0.53 | 7 |
| 017 | 9 | 0.43 | 5 |
| 018 | 9 | 0.47 | 4 |
| 019 | 9 | 0.42 | 4 |
| 020 | 9 | 0.27 | 1 |
| Number | H-bond acceptors | H-bond donors | MR |
| 001 | 2 | 3 | 85.31 |
| 002 | 3 | 2 | 102.96 |
| 003 | 3 | 2 | 96.03 |
| 004 | 3 | 2 | 96.03 |
| 005 | 2 | 3 | 102.55 |
| 006 | 3 | 3 | 80.19 |
| 007 | 3 | 2 | 73.36 |
| 008 | 2 | 2 | 90.96 |
| 009 | 4 | 2 | 105.85 |
| 010 | 4 | 3 | 99.15 |
| 011 | 4 | 2 | 97.43 |
| 012 | 3 | 3 | 104.33 |
| 013 | 4 | 3 | 90.01 |
| 014 | 4 | 2 | 96.55 |
| 015 | 4 | 2 | 105.85 |
| 016 | 4 | 2 | 108.88 |
| 017 | 0 | 2 | 71.45 |
| 018 | 0 | 2 | 76.26 |
| 019 | 1 | 1 | 63.05 |
| 020 | 0 | 2 | 54.73 |
| Number | TPSA | LOGP | XLOGP3 |
| 001 | 58.14 | 2.04 | 2.72 |
| 002 | 41.93 | 3.58 | 3.26 |
| 003 | 41.93 | 3.08 | 2.81 |
| 004 | 41.93 | 2.91 | 2.81 |
| 005 | 58.56 | 2.9 | 3.85 |
| 006 | 58.92 | 2.83 | 2.05 |
| 007 | 69.59 | 2.02 | 0.26 |
| 008 | 42.35 | 2.86 | 3.51 |
| 009 | 59 | 3.11 | 2.94 |
| 010 | 67.79 | 2.77 | 2.64 |
| 011 | 64.99 | 3.06 | 2.92 |
| 012 | 66.38 | 3.14 | 2.62 |
| 013 | 75.99 | 2.83 | 2.18 |
| 014 | 59 | 3.21 | 2.55 |
| 015 | 59 | 3.11 | 2.94 |
| 016 | 53.01 | 3.21 | 2.51 |
| 017 | 20.23 | 2.78 | 2.57 |
| 018 | 20.23 | 2.73 | 3.52 |
| 019 | 37.79 | 2.32 | 2.36 |
| 020 | 43.43 | 1.64 | 1.36 |
| Number | WLOGP | MLOGP | Consensus Log P |
| 001 | 1.76 | −2.63 | 1.44 |
| 002 | 1.27 | −1.57 | 1.99 |
| 003 | 0.63 | −1.81 | 1.45 |
| 004 | 0.63 | −1.81 | 1.42 |
| 005 | 2.67 | −0.99 | 2.38 |
| 006 | 0.55 | −2.54 | 1.08 |
| 007 | −0.03 | −2.47 | 0.38 |
| 008 | 2.43 | −1.36 | 2.25 |
| 009 | 0.94 | −1.43 | 1.65 |
| 010 | 1.22 | −1.66 | 1.59 |
| 011 | 1.65 | −1.66 | 1.85 |
| 012 | −0.03 | −5.34 | 0.62 |
| 013 | 0.85 | −2.13 | 1.32 |
| 014 | 1.03 | −1.89 | 1.51 |
| 015 | 0.94 | −1.43 | 1.65 |
| 016 | −0.06 | −1.66 | 1.25 |
| 017 | 1.64 | −1.31 | 1.83 |
| 018 | 2.02 | −1.06 | 2.15 |
| 019 | 2.52 | 1.55 | 2.33 |
| 020 | 1.27 | −2.13 | 0.91 |
| | | ESOL Solubility | ESOL Solubility |
| Number | ESOL Log S | (mg/ml) | (mol/l) |
| 001 | −3.42 | 0.108 | 0.00038 |
| 002 | −3.64 | 0.0764 | 0.00023 |
| 003 | −3.47 | 0.108 | 0.000342 |
| 004 | −3.47 | 0.108 | 0.000342 |
| 005 | −4.28 | 0.0178 | 0.0000525 |
| 006 | −2.67 | 0.599 | 0.00215 |
| 007 | −1.58 | 6.83 | 0.026 |
| 008 | −4 | 0.0298 | 0.000101 |
| 009 | −3.71 | 0.0701 | 0.000196 |
| 010 | −3.39 | 0.141 | 0.000408 |
| 011 | −3.57 | 0.0931 | 0.000268 |
| 012 | −3.19 | 0.224 | 0.000643 |
| 013 | −2.84 | 0.469 | 0.00146 |
| 014 | −3.14 | 0.244 | 0.000729 |
| 015 | −3.71 | 0.0701 | 0.000196 |
| 016 | −3.37 | 0.148 | 0.000429 |
| 017 | −2.89 | 0.278 | 0.00128 |
| 018 | −3.62 | 0.0555 | 0.00024 |
| 019 | −2.77 | 0.344 | 0.00169 |
| 020 | −2.22 | 1.05 | 0.00607 |
| | Solubility | (II-method) | Solubility |
| Number | Class (ESOL) | Log S | (mg/ml) |
| 001 | Soluble | −3.59 | 0.0726 |
| 002 | Soluble | −3.81 | 0.0509 |
| 003 | Soluble | −3.35 | 0.142 |
| 004 | Soluble | −3.35 | 0.142 |
| 005 | Moderately soluble | −4.78 | 0.00567 |
| 006 | Soluble | −2.92 | 0.339 |
| 007 | Very soluble | −1.28 | 13.7 |
| 008 | Soluble | −4.08 | 0.0245 |
| 009 | Soluble | −3.84 | 0.0517 |
| 010 | Soluble | −3.71 | 0.0669 |
| 011 | Soluble | −3.95 | 0.0393 |
| 012 | Soluble | −3.66 | 0.0755 |
| 013 | Soluble | −3.41 | 0.125 |
| 014 | Soluble | −3.44 | 0.122 |
| 015 | Soluble | −3.84 | 0.0517 |
| 016 | Soluble | −3.27 | 0.186 |
| 017 | Soluble | −2.64 | 0.495 |
| 018 | Soluble | −3.63 | 0.0544 |
| 019 | Soluble | −2.79 | 0.328 |
| 020 | Soluble | −1.87 | 2.31 |
| Number | Solubility (mol/l) | II method Class | Pgp substrate |
| 001 | 0.000254 | Soluble | Yes |
| 002 | 0.000153 | Soluble | No |
| 003 | 0.000449 | Soluble | No |
| 004 | 0.000449 | Soluble | No |
| 005 | 0.0000167 | Moderately soluble | No |
| 006 | 0.00121 | Soluble | Yes |
| 007 | 0.0522 | Very soluble | No |
| 008 | 0.0000826 | Moderately soluble | No |
| 009 | 0.000144 | Soluble | No |
| 010 | 0.000193 | Soluble | Yes |
| 011 | 0.000113 | Soluble | Yes |
| 012 | 0.000217 | Soluble | Yes |
| 013 | 0.00039 | Soluble | Yes |
| 014 | 0.000366 | Soluble | No |
| 015 | 0.000144 | Soluble | No |
| 016 | 0.000538 | Soluble | Yes |
| 017 | 0.00228 | Soluble | No |
| 018 | 0.000235 | Soluble | No |
| 019 | 0.00161 | Soluble | No |
| 020 | 0.0134 | Very soluble | No |
| | CYP1A2 | CYP2C19 | CYP2C9 |
| Number | inhibitor | inhibitor | inhibitor |
| 001 | Yes | No | No |
| 002 | No | No | No |
| 003 | No | No | No |
| 004 | No | No | No |
| 005 | Yes | No | No |
| 006 | No | No | No |
| 007 | No | No | No |
| 008 | Yes | No | No |
| 009 | No | No | No |
| 010 | No | No | No |
| 011 | No | No | No |
| 012 | No | No | No |
| 013 | No | No | No |
| 014 | No | No | No |
| 015 | No | No | No |
| 016 | No | No | No |
| 017 | No | No | No |
| 018 | Yes | No | No |
| 019 | Yes | Yes | No |
| 020 | Yes | No | No |
| | CYP2D6 | CYP3A4 | |
| Number | inhibitor | inhibitor | log Kp (cm/s) |
| 001 | Yes | No | −6.11 |
| 002 | No | No | −6.01 |
| 003 | No | No | −6.24 |
| 004 | No | No | −6.24 |
| 005 | Yes | No | −5.63 |
| 006 | No | No | −6.55 |
| 007 | No | No | −7.72 |
| 008 | Yes | No | −5.62 |
| 009 | No | No | −6.4 |
| 010 | No | No | −6.54 |
| 011 | No | No | −6.35 |
| 012 | No | No | −6.57 |
| 013 | No | No | −6.71 |
| 014 | No | No | −6.53 |
| 015 | No | No | −6.4 |
| 016 | No | No | −6.63 |
| 017 | No | No | −5.8 |
| 018 | No | No | −5.21 |
| 019 | No | No | −5.87 |
| 020 | No | No | −6.39 |
| | Lipinski | Ghose | Veber |
| Number | #violations | violations | violations |
| 001 | 0 | 0 | 0 |
| 002 | 0 | 0 | 0 |
| 003 | 0 | 0 | 0 |
| 004 | 0 | 0 | 0 |
| 005 | 0 | 0 | 0 |
| 006 | 0 | 0 | 0 |
| 007 | 0 | 0 | 0 |
| 008 | 0 | 0 | 0 |
| 009 | 0 | 0 | 0 |
| 010 | 0 | 0 | 0 |
| 011 | 0 | 0 | 0 |
| 012 | 0 | 0 | 1 |
| 013 | 0 | 0 | 0 |
| 014 | 0 | 0 | 0 |
| 015 | 0 | 0 | 0 |
| 016 | 0 | 0 | 0 |
| 017 | 0 | 0 | 0 |
| 018 | 0 | 0 | 0 |
| 019 | 0 | 0 | 0 |
| 020 | 0 | 0 | 0 |
| | Egan | Muegge | Bioavailability |
| Number | violations | violations | Score |
| 001 | 0 | 0 | 0.55 |
| 002 | 0 | 0 | 0.55 |
| 003 | 0 | 0 | 0.55 |
| 004 | 0 | 0 | 0.55 |
| 005 | 0 | 0 | 0.55 |
| 006 | 0 | 0 | 0.55 |
| 007 | 0 | 0 | 0.55 |
| 008 | 0 | 0 | 0.55 |
| 009 | 0 | 0 | 0.55 |
| 010 | 0 | 0 | 0.55 |
| 011 | 0 | 0 | 0.55 |
| 012 | 0 | 0 | 0.55 |
| 013 | 0 | 0 | 0.55 |
| 014 | 0 | 0 | 0.55 |
| 015 | 0 | 0 | 0.55 |
| 016 | 0 | 0 | 0.55 |
| 017 | 0 | 0 | 0.55 |
| 018 | 0 | 0 | 0.55 |
| 019 | 0 | 0 | 0.55 |
| 020 | 0 | 1 | 0.55 |
| | PAINS | | Leadlikeness |
| Number | alerts | Brenk alerts | violations |
| 001 | 0 | 0 | 0 |
| 002 | 0 | 1 | 1 |
| 003 | 0 | 0 | 0 |
| 004 | 0 | 0 | 0 |
| 005 | 0 | 0 | 2 |
| 006 | 0 | 1 | 1 |
| 007 | 0 | 1 | 0 |
| 008 | 0 | 0 | 1 |
| 009 | 0 | 1 | 1 |
| 010 | 0 | 1 | 1 |
| 011 | 0 | 2 | 1 |
| 012 | 0 | 2 | 1 |
| 013 | 0 | 2 | 1 |
| 014 | 0 | 2 | 1 |
| 015 | 0 | 1 | 1 |
| 016 | 0 | 1 | 0 |
| 017 | 0 | 0 | 1 |
| 018 | 0 | 0 | 2 |
| 019 | 0 | 0 | 1 |
| 020 | 0 | 0 | 1 |

| Number | Synthetic Accessibility | Docking scores (Kcal/mol) |
|---|---|---|
| 001 | 2.79 | −12.691 |
| 002 | 3.36 | −11.829 |
| 003 | 4.67 | −11.791 |
| 004 | 4.61 | −11.629 |
| 005 | 2.88 | −11.176 |
| 006 | 2.95 | −11.051 |
| 007 | 2.27 | −10.691 |
| 008 | 2.76 | −10.657 |
| 009 | 4.68 | −12.441 |
| 010 | 3.09 | −11.148 |
| 011 | 3.17 | −11.018 |
| 012 | 3.15 | −10.88 |
| 013 | 3 | −10.628 |
| 014 | 3.13 | −10.027 |
| 015 | 4.68 | −9.917 |
| 016 | 3.19 | −9.824 |
| 017 | 1.82 | −10.463 |
| 018 | 2.47 | −9.724 |
| 019 | 1.78 | −5.853 |
| 020 | 2.35 | −8.579 |

### Naming and Strings

IUPAC name=compound name.
Smile=Smile naming convention of compound
Formula=Chemical formula of compound

### Physiochemical Properties

MW=Molecular weight
Heavy atoms=Atoms with significantly higher atomic scattering factor than the others present
Aromatic heavy atoms=As above referring to the ring structures
Csp3=the ratio of sp3 hybridized carbons over the total carbon count of the molecule (> or equal to 0.25)
Rotable bonds=Bonds in the molecule that can rotate
H-Bonds acceptor=Bonds that can accept hydrogen ion
H-Bonds donors=Bonds that can donate hydrogen ion
MR=Molecular refractivity
TPSA=topological polar surface area

### Lipophilicity

LOGP=partition coefficient for ionisable compounds. An approximation implemented by CHARMM version c36 (Chemistry at Harvard Macromolecular Mechanics)
XLOGP=another atomistic method with correction factors from: Cheng, T. et al. Computation of Octanol-Water Partition Coefficients by Guiding an Additive Model with Knowledge. J Chem Inf. Model 47, 2140-2148 (2007).
WLOGP=is another LogP using the Wildman method described in: Wildman, S. A. & Crippen, G. M. Prediction of Physicochemical Parameters by Atomic Contributions. J. Chem. Inf. Model. 39,868-873 (1999).
MLOGP=Moriguchi topological method for partition coefficient. Moriguchi, I., Shuichi, H., Liu, Q., Nakagome, I. & Matsushita, Y. Simple Method of Calculating Octanol/Water Partition Coefficient. Chem. Pharm. Bull. 40, 127-130 (1992).
General Log p=In order to increase the accuracy of the Log P o/W the above methods were used and a general estimation of these values was condensed in “General Log P” column

### Solubility

ESOL Log S=Aqueous solubility by ESOL method: Delaney, J. S. ESOL: Estimating Aqueous Solubility Directly from Molecular Structure. J. Chem. Inf. Model. 44, 1000-1005 (2004)
ESOL Solubility (mg/ml)=quantification of solubility by SwissADME
ESOL Solubility (mol/l)=as above
Solubility class for ESOL method=solubility in aqueous solution
II methods Log S=Solubility method based on: Ali, J., Camilleri, P., Brown, M. B., Hutt, A. J. & Kirton, S. B. Revisiting the general solubility equation: in silico prediction of aqueous solubility incorporating the effect of topographical polar surface area. J. Chem. Inf. Model. 52, 420-428 (2012).

### Pharmacokinetics

Pgp substrate=P glycoprotein, this describes if the compound is a substrate of glycoprotein associated with the permeability of biological membranes.
The below subfamilies of the cytochrome P450 determine drug elimination and metabolism in association with Pgp data: CYP1A2 inhibitor CYP2C19 inhibitor, CYP2C9 inhibitor, CYP2D6 inhibitor, CYP3A4 inhibitor,

### Drug-Likeness

Lipinski violations
Ghose violations
Veber violations
Egan violations
Muegge violations
Bioavailability Score (The Abbot Bioavailability Score)

### Synthesis

“PAINS (Pan-assay interference compounds)=Baell, J. B. & Holloway, G. A. New substructure filters for removal of pan assay interference compounds (PAINS) from screening libraries and for their exclusion in bioassays. J. Med. Chem. 53, 2719-2740 (2010).”
“Brenk alerts=Brenk, R. et al. Lessons learnt from assembling screening libraries for drug discovery for neglected diseases. ChemMedChem 3,435-444 (2008).”
“Lead likeness violations=based on: Teague, S., Davis, A., Leeson, P. & Oprea, T. The Design of Lead like Combinatorial Libraries. Angew. Chem. Int. Ed. Engl. 38,3743-3748 (1999).”
“Synthetic Accessibility=based on two papers: Fukunishi, Y., Kurosawa, T., Mikami, Y. & Nakamura, H. Prediction of synthetic accessibility based on commercially available compound databases. J Chem Inf Model 54, 3259-3267 (2014).
Ertl, P. & Schuffenhauer, A. Estimation of synthetic accessibility score of drug-like molecules based on molecular complexity and fragment contributions. J. Cheminform. 1, 8 (2009). From 1 to 10 with 1 easy and 10 complex”

### Docking Scores (Kcal/Mol)

Scores are reported for docking to the target, the highest negative number indicates a better binding pose of the ligand in the receptor.

### Abbreviations

| | |
|---|---|
| 5HTR# = 5-hydroxytryptamine | MTOR = Mu-type opioid |
| receptor # | receptor |
| ACHR =Acetylcholinesterase | NaClDGT# = Sodium and |
| B#AR = Beta-# adrenergic | chloride-dependent |
| receptor | glycine transporter # |
| CHR = Cholinesterase | NaDDT = Sodium-dependent |
| CP45011# = Cytochrome | dopamine transporter |
| P450 11# | NaDNT = Sodium-dependent |
| D(#)DR = D(#) dopamine receptor | noradrenaline transporter |
| Enz = Enzyme | NaDPT = Sodium-dependent |
| FAAH1 = Fatty-acid amide | proline transporter |
| hydrolase 1 | NaDST = Sodium-dependent |
| M.Rec = Membrane receptor | serotonin transporter |
| MARM# = Muscarinic | TDNAP1 = Tyrosyl-DNA |
| acetylcholine receptor M# | phosphodiesterase 1 |
| MBLP# = Muscleblind-like | TPKJAK# = Tyrosine-protein |
| protein # | kinase JAK# |
| Mitchond = mitochondrial | Trans = Transporter |
| MRT1A = Melatonin receptor | Unc = Unclassified |
| type 1A | where # = a number |

### Compound/Target Data

### Further Information Regarding Targets Screened

The table below details the range of targets that selected tryptamine analogues were screened against and the results.

| | | | | | | | Number | Number | |
|---|---|---|---|---|---|---|---|---|---|
| | | | | | | | simulate | simulated | |
| Compound | | | | | By | | compounds | compounds | Target |
| number. | Target | Uniprot ID | Gene Code | ChEMBL ID | Homology | Probability | (3D) | (2D) | Class |
| 001 | 5HTR6 | P50406 | HTR6 | 3371 | No | 0.48 | 584 | 37 | M.Rec |
| | 5HTR2A | P28223 | HTR2A | 224 | No | 0.42 | 486 | 99 | M.Rec |
| | 5HTR1A | P08908 | HTR1A | 214 | No | 0.4 | 649 | 174 | M.Rec |
| | 5HTR1B | P28222 | HTR1B | 1898 | Yes | 0.4 | 699 | 179 | M.Rec |
| | 5HTR2C | P28335 | HTR2C | 225 | No | 0.38 | 321 | 67 | M.Rec |
| | 5HTR2B | P41595 | HTR2B | 1833 | No | 0.38 | 321 | 67 | M.Rec |
| | CHR | P06276 | BCHE | 1914 | No | 0.34 | 228 | 3 | Enz. |
| | ACHR | P22303 | ACHE | 220 | No | 0.34 | 228 | 3 | Enz. |
| | 5HTR1D | P28221 | HTR1D | 1983 | No | 0.34 | 270 | 129 | M.Rec |
| | 5HTR1E | P28566 | HTR1E | 2182 | Yes | 0.32 | 210 | 124 | M.Rec |
| | 5HTR1F | P30939 | HTR1F | 1805 | Yes | 0.32 | 210 | 124 | M.Rec |
| | 5HTR7 | P34969 | HTR7 | 3155 | No | 0.32 | 198 | 15 | M.Rec |
| | D(2)DR | P14416 | DRD2 | 217 | No | 0.27 | 875 | 23 | M.Rec |
| | D(4)DR | P21917 | DRD4 | 219 | No | 0.27 | 366 | 13 | M.Rec |
| | D(3)DR | P35462 | DRD3 | 234 | No | 0.27 | 571 | 18 | M.Rec |
| 002 | 5HTR2A | P28223 | HTR2A | 224 | No | 0.47 | 115 | 97 | M.Rec |
| | 5HTR2C | P28335 | HTR2C | 225 | No | 0.47 | 90 | 75 | M.Rec |
| | 5HTR2B | P41595 | HTR2B | 1833 | No | 0.47 | 90 | 75 | M.Rec |
| | 5HTR1A | P08908 | HTR1A | 214 | No | 0.38 | 85 | 206 | M.Rec |
| | 5HTR1B | P28222 | HTR1B | 1898 | No | 0.38 | 88 | 211 | M.Rec |
| | 5HTR6 | P50406 | HTR6 | 3371 | No | 0.33 | 203 | 46 | M.Rec |
| | 5HTR1D | P28221 | HTR1D | 1983 | No | 0.27 | 40 | 168 | M.Rec |
| | 5HTR1E | P28566 | HTR1E | 2182 | Yes | 0.27 | 36 | 163 | M.Rec |
| | 5HTR1F | P30939 | HTR1F | 1805 | Yes | 0.27 | 36 | 163 | M.Rec |
| | 5HTR7 | P34969 | HTR7 | 3155 | No | 0.26 | 54 | 11 | M.Rec |
| | TDNAP1 | Q9NUW8 | TDP1 | 1075138 | No | 0.24 | 38 | 3 | Enz. |
| | NaDST | P31645 | SLC6A4 | 228 | Yes | 0.22 | 243 | 33 | Trans |
| | CHR | P06276 | BCHE | 1914 | No | 0.21 | 85 | 4 | Enz. |
| | ACHR | P22303 | ACHE | 220 | No | 0.21 | 85 | 4 | Enz. |
| | D(2)DR | P14416 | DRD2 | 217 | No | 0.21 | 108 | 47 | M.Rec |
| 003 | 5HTR2A | P28223 | HTR2A | 224 | No | 0.28 | 268 | 49 | M.Rec |
| | 5HTR2C | P28335 | HTR2C | 225 | No | 0.28 | 204 | 34 | M.Rec |
| | 5HTR2B | P41595 | HTR2B | 1833 | No | 0.28 | 204 | 34 | M.Rec |
| | 5HTR1B | P28222 | HTR1B | 1898 | Yes | 0.28 | 287 | 101 | M.Rec |
| | 5HTR1D | P28221 | HTR1D | 1983 | No | 0.28 | 84 | 61 | M.Rec |
| | 5HTR1A | P08908 | HTR1A | 214 | No | 0.28 | 282 | 96 | M.Rec |
| | 5HTR1E | P28566 | HTR1E | 2182 | Yes | 0.25 | 73 | 56 | M.Rec |
| | 5HTR1F | P30939 | HTR1F | 1805 | Yes | 0.25 | 73 | 56 | M.Rec |
| | D(2)DR | P14416 | DRD2 | 217 | No | 0.25 | 520 | 4 | M.Rec |
| | NaDST | P31645 | SLC6A4 | 228 | Yes | 0.23 | 862 | 38 | Trans |
| | NaDNT | P23975 | SLC6A2 | 222 | Yes | 0.23 | 854 | 17 | Trans |
| | NaDDT | Q01959 | SLC6A3 | 238 | Yes | 0.23 | 854 | 17 | Trans |
| | NaCIDGT1 | P48067 | SLC6A9 | 2337 | Yes | 0.23 | 586 | 16 | Trans |
| | NaCIDGT2 | Q9Y345 | SLC6A5 | 3060 | Yes | 0.23 | 586 | 16 | Trans |
| | NaDPT | Q99884 | SLC6A7 | | Yes | 0.23 | 586 | 16 | Trans |
| 004 | 5HTR2A | P28223 | HTR2A | 224 | No | 0.32 | 280 | 70 | M.Rec |
| | 5HTR2C | P28335 | HTR2C | 225 | No | 0.32 | 213 | 49 | M.Rec |
| | 5HTR2B | P41595 | HTR2B | 1833 | No | 0.32 | 213 | 49 | M.Rec |
| | 5HTR1A | P08908 | HTR1A | 214 | No | 0.3 | 307 | 137 | M.Rec |
| | 5HTR1B | P28222 | HTR1B | 1898 | Yes | 0.3 | 312 | 142 | M.Rec |
| | 5HTR6 | P50406 | HTR6 | 3371 | No | 0.28 | 74 | 31 | M.Rec |
| | 5HTR1D | P28221 | HTR1D | 1983 | No | 0.25 | 90 | 102 | M.Rec |
| | MARM4 | P08173 | CHRM4 | 1821 | Yes | 0.25 | 267 | 2 | M.Rec |
| | MARM5 | P08912 | CHRM5 | 2035 | Yes | 0.25 | 222 | 2 | M.Rec |
| | MARM1 | P11229 | CHRM1 | 216 | No | 0.25 | 308 | 3 | M.Rec |
| | MARM3 | P20309 | CHRM3 | 245 | No | 0.25 | 251 | 2 | M.Rec |
| | 5HTR1E | P28566 | HTR1E | 2182 | Yes | 0.23 | 79 | 97 | M.Rec |
| | 5HTR1F | P30939 | HTR1F | 1805 | Yes | 0.23 | 79 | 97 | M.Rec |
| | NaDST | P31645 | SLC6A4 | 228 | Yes | 0.23 | 991 | 39 | Trans |
| | D(2)DR | P14416 | DRD2 | 217 | No | 0.23 | 567 | 14 | M.Rec |
| 005 | 5HTR6 | P50406 | HTR6 | 3371 | No | 0.48 | 337 | 51 | M.Rec |
| | FAAH1 | O00519 | FAAH | 2243 | Yes | 0.43 | 13 | 19 | Enz. |
| | 5HTR2A | P28223 | HTR2A | 224 | No | 0.41 | 197 | 91 | M.Rec |
| | 5HTR1A | P08908 | HTR1A | 214 | No | 0.39 | 256 | 174 | M.Rec |
| | 5HTR1B | P28222 | HTR1B | 1898 | No | 0.39 | 271 | 182 | M.Rec |
| | 5HTR2C | P28335 | HTR2C | 225 | Yes | 0.37 | 148 | 67 | M.Rec |
| | 5HTR2B | P41595 | HTR2B | 1833 | Yes | 0.37 | 148 | 67 | M.Rec |
| | CHR | P06276 | BCHE | 1914 | Yes | 0.37 | 146 | 215 | Enz. |
| | ACHR | P22303 | ACHE | 220 | Yes | 0.37 | 146 | 215 | Enz. |
| | 5HTR1D | P28221 | HTR1D | 1983 | No | 0.37 | 113 | 147 | M.Rec |
| | 5HTR1E | P28566 | HTR1E | 2182 | Yes | 0.37 | 95 | 139 | M.Rec |
| | 5HTR1F | P30939 | HTR1F | 1805 | Yes | 0.37 | 95 | 139 | M.Rec |
| | 5HTR7 | P34969 | HTR7 | 3155 | No | 0.29 | 92 | 18 | M.Rec |
| | NaDNT | P23975 | SLC6A2 | 222 | No | 0.26 | 535 | 10 | Trans |
| | NaDDT | Q01959 | SLC6A3 | 238 | Yes | 0.26 | 535 | 10 | Trans |
| 006 | 5HTR1B | P28222 | HTR1B | 1898 | Yes | 0.66 | 194 | 179 | M.Rec |
| | 5HTR2A | P28223 | HTR2A | 224 | No | 0.66 | 162 | 115 | M.Rec |
| | 5HTR1A | P08908 | HTR1A | 214 | No | 0.62 | 186 | 174 | M.Rec |
| | 5HTR2C | P28335 | HTR2C | 225 | No | 0.6 | 110 | 87 | M.Rec |
| | 5HTR2B | P41595 | HTR2B | 1833 | No | 0.6 | 110 | 87 | M.Rec |
| | 5HTR1D | P28221 | HTR1D | 1983 | Yes | 0.56 | 55 | 141 | M.Rec |
| | 5HTR6 | P50406 | HTR6 | 3371 | No | 0.54 | 46 | 53 | M.Rec |
| | NaDST | P31645 | SLC6A4 | 228 | No | 0.5 | 572 | 18 | Trans |
| | 5HTR1E | P28566 | HTR1E | 2182 | Yes | 0.44 | 43 | 135 | M.Rec |
| | 5HTR1F | P30939 | HTR1F | 1805 | No | 0.44 | 43 | 135 | M.Rec |
| | TDNAP1 | Q9NUW8 | TDP1 | 1075138 | No | 0.44 | 96 | 5 | Enz. |
| | D(2)DR | P14416 | DRD2 | 217 | No | 0.44 | 338 | 53 | M.Rec |
| | D(1A)DR | P21728 | DRD1 | 2056 | No | 0.44 | 33 | 13 | M.Rec |
| | D(4)DR | P21917 | DRD4 | 219 | Yes | 0.44 | 195 | 16 | M.Rec |
| | D(1B)DR | P21918 | DRD5 | 1850 | Yes | 0.44 | 28 | 13 | M.Rec |
| 007 | 5HTR2A | P28223 | HTR2A | 224 | Yes | 0.5 | 6 | 152 | M.Rec |
| | CHR | P06276 | BCHE | 1914 | Yes | 0.5 | 11 | 15 | Enz. |
| | ACHR | P22303 | ACHE | 220 | No | 0.5 | 11 | 15 | Enz. |
| | NaDNT | P23975 | SLC6A2 | 222 | No | 0.44 | 10 | 40 | Trans |
| | NaDDT | Q01959 | SLC6A3 | 238 | Yes | 0.44 | 10 | 40 | Trans |
| | 5HTR1A | P08908 | HTR1A | 214 | No | 0.38 | 4 | 255 | M.Rec |
| | 5HTR1B | P28222 | HTR1B | 1898 | Yes | 0.38 | 4 | 262 | M.Rec |
| | TDNAP1 | Q9NUW8 | TDP1 | 1075138 | No | 0.37 | 17 | 5 | Enz. |
| | 5HTR2C | P28335 | HTR2C | 225 | Yes | 0.37 | 2 | 109 | M.Rec |
| | 5HTR2B | P41595 | HTR2B | 1833 | No | 0.37 | 2 | 109 | M.Rec |
| | FAAH1 | O00519 | FAAH | 2243 | No | 0.33 | 7 | 12 | Enz. |
| | B2AR | P07550 | ADRB2 | 210 | No | 0.33 | 28 | 13 | M.Rec |
| | B1AR | P08588 | ADRB1 | 213 | Yes | 0.33 | 33 | 13 | M.Rec |
| | B3AR | P13945 | ADRB3 | 246 | Yes | 0.33 | 26 | 13 | M.Rec |
| | 5HTR1D | P28221 | HTR1D | 1983 | No | 0.33 | 1 | 189 | M.Rec |
| 008 | 5HTR6 | P50406 | HTR6 | 3371 | No | 0.59 | 282 | 73 | M.Rec |
| | 5HTR2A | P28223 | HTR2A | 224 | Yes | 0.5 | 181 | 128 | M.Rec |
| | 5HTR1A | P08908 | HTR1A | 214 | No | 0.48 | 143 | 211 | M.Rec |
| | 5HTR1B | P28222 | HTR1B | 1898 | No | 0.48 | 151 | 217 | M.Rec |
| | 5HTR2C | P28335 | HTR2C | 225 | No | 0.44 | 149 | 91 | M.Rec |
| | 5HTR2B | P41595 | HTR2B | 1833 | No | 0.44 | 149 | 91 | M.Rec |
| | 5HTR1D | P28221 | HTR1D | 1983 | No | 0.42 | 57 | 144 | M.Rec |
| | 5HTR1E | P28566 | HTR1E | 2182 | Yes | 0.4 | 47 | 137 | M.Rec |
| | 5HTR1F | P30939 | HTR1F | 1805 | No | 0.4 | 47 | 137 | M.Rec |
| | 5HTR7 | P34969 | HTR7 | 3155 | No | 0.4 | 58 | 20 | M.Rec |
| | NaDNT | P23975 | SLC6A2 | 222 | No | 0.34 | 535 | 70 | Trans |
| | NaDST | P31645 | SLC6A4 | 228 | Yes | 0.34 | 486 | 105 | Trans |
| | NaDDT | Q01959 | SLC6A3 | 238 | Yes | 0.34 | 535 | 70 | Trans |
| | NaCIDGT1 | P48067 | SLC6A9 | 2337 | Yes | 0.34 | 382 | 69 | Trans |
| | NaDPT | Q99884 | SLC6A7 | | Yes | 0.34 | 382 | 69 | Trans |
| 009 | 5HTR1A | P08908 | HTR1A | 214 | No | 0.33 | 296 | 109 | M.Rec |
| | 5HTR1B | P28222 | HTR1B | 1898 | No | 0.33 | 301 | 110 | M.Rec |
| | 5HTR1D | P28221 | HTR1D | 1983 | No | 0.31 | 91 | 62 | M.Rec |
| | 5HTR1E | P28566 | HTR1E | 2182 | Yes | 0.31 | 80 | 61 | M.Rec |
| | 5HTR1F | P30939 | HTR1F | 1805 | Yes | 0.31 | 80 | 61 | M.Rec |
| | MARM4 | P08173 | CHRM4 | 1821 | Yes | 0.29 | 235 | 2 | M.Rec |
| | MARM5 | P08912 | CHRM5 | 2035 | Yes | 0.29 | 195 | 2 | M.Rec |
| | MARM1 | P11229 | CHRM1 | 216 | No | 0.29 | 232 | 3 | M.Rec |
| | MARM3 | P20309 | CHRM3 | 245 | No | 0.29 | 224 | 2 | M.Rec |
| | NaDNT | P23975 | SLC6A2 | 222 | Yes | 0.27 | 776 | 22 | Trans |
| | NaDST | P31645 | SLC6A4 | 228 | No | 0.27 | 802 | 46 | Trans |
| | NaCIDGT1 | P48067 | SLC6A9 | 2337 | Yes | 0.27 | 554 | 21 | Trans |
| | NaDDT | Q01959 | SLC6A3 | 238 | Yes | 0.27 | 776 | 22 | Trans |
| | NaCIDGT2 | Q9Y345 | SLC6A5 | 3060 | Yes | 0.27 | 554 | 21 | Trans |
| | NaDPT | Q99884 | SLC6A7 | | Yes | 0.27 | 554 | 21 | Trans |
| 010 | 5HTR1A | P08908 | HTR1A | 214 | No | 0.48 | 311 | 166 | M.Rec |
| | 5HTR1B | P28222 | HTR1B | 1898 | Yes | 0.48 | 317 | 171 | M.Rec |
| | 5HTR6 | P50406 | HTR6 | 3371 | No | 0.46 | 61 | 32 | M.Rec |
| | 5HTR1D | P28221 | HTR1D | 1983 | No | 0.46 | 100 | 129 | M.Rec |
| | 5HTR1E | P28566 | HTR1E | 2182 | Yes | 0.46 | 88 | 124 | M.Red |
| | 5HTR1F | P30939 | HTR1F | 1805 | Yes | 0.46 | 88 | 124 | M.Rec |
| | B2AR | P07550 | ADRB2 | 210 | No | 0.39 | 481 | 6 | M.Rec |
| | B1AR | P08588 | ADRB1 | 213 | Yes | 0.39 | 482 | 6 | M.Rec |
| | B3AR | P13945 | ADRB3 | 246 | No | 0.39 | 462 | 6 | M.Rec |
| | 5HTR2A | P28223 | HTR2A | 224 | No | 0.37 | 275 | 96 | M.Red |
| | 5HTR2C | P28335 | HTR2C | 225 | No | 0.35 | 209 | 69 | M.Rec |
| | 5HTR2B | P41595 | HTR2B | 1833 | No | 0.35 | 209 | 69 | M . Rec |
| | 5HTR7 | P34969 | HTR7 | 3155 | No | 0.31 | 47 | 11 | M.Rec |
| | NaDST | P31645 | SLC6A4 | 228 | Yes | 0.31 | 863 | 30 | Trans |
| | MRT1A | P48039 | MTNR1A | 1945 | No | 0.31 | 13 | 83 | M.Rec |
| 011 | 5HTR1A | P08908 | HTR1A | 214 | Yes | 0.37 | 266 | 194 | M.Rec |
| | 5HTR1D | P28221 | HTR1D | 1983 | No | 0.37 | 79 | 141 | M.Rec |
| | 5HTR1B | P28222 | HTR1B | 1898 | No | 0.37 | 273 | 200 | M.Rec |
| | 5HTR1E | P28566 | HTR1E | 2182 | Yes | 0.37 | 66 | 135 | M.Rec |
| | 5HTR1F | P30939 | HTR1F | 1805 | Yes | 0.37 | 66 | 135 | M.Rec |
| | 5HTR6 | P50406 | HTR6 | 3371 | No | 0.37 | 46 | 33 | M.Rec |
| | 5HTR2A | P28223 | HTR2A | 224 | Yes | 0.31 | 205 | 89 | M.Rec |
| | 5HTR2C | P28335 | HTR2C | 225 | No | 0.28 | 146 | 66 | M.Rec |
| | 5HTR2B | P41595 | HTR2B | 1833 | No | 0.28 | 146 | 66 | M.Red |
| | 5HTR7 | P34969 | HTR7 | 3155 | No | 0.26 | 31 | 12 | M.Rec |
| | B2AR | P07550 | ADRB2 | 210 | No | 0.24 | 433 | 3 | M.Rec |
| | B1AR | P08588 | ADRB1 | 213 | No | 0.24 | 439 | 3 | M.Rec |
| | B3AR | P13945 | ADRB3 | 246 | Yes | 0.24 | 425 | 3 | M.Rec |
| | NaDNT | P23975 | SLC6A2 | 222 | No | 0.24 | 366 | 24 | Trans |
| | NaDST | P31645 | SLC6A4 | 228 | No | 0.24 | 421 | 50 | Trans |
| 012 | 5HTR1A | P08908 | HTR1A | 214 | Yes | 0.43 | 379 | 202 | M.Rec |
| | 5HTR1D | P28221 | HTR1D | 1983 | No | 0.43 | 159 | 153 | M.Rec |
| | 5HTR1B | P28222 | HTR1B | 1898 | No | 0.43 | 399 | 208 | M.Rec |
| | 5HTR1E | P28566 | HTR1E | 2182 | Yes | 0.43 | 132 | 146 | M.Rec |
| | 5HTR1F | P30939 | HTR1F | 1805 | Yes | 0.43 | 132 | 146 | M.Rec |
| | 5HTR6 | P50406 | HTR6 | 3371 | No | 0.37 | 175 | 48 | M.Rec |
| | 5HTR2A | P28223 | HTR2A | 224 | Yes | 0.31 | 223 | 115 | M.Rec |
| | 5HTR2C | P28335 | HTR2C | 225 | No | 0.24 | 136 | 86 | M.Rec |
| | 5HTR2B | P41595 | HTR2B | 1833 | No | 0.24 | 136 | 86 | M.Rec |
| | B2AR | P07550 | ADRB2 | 210 | No | 0.24 | 16 | 7 | M.Rec |
| | B1AR | P08588 | ADRB1 | 213 | No | 0.24 | 19 | 7 | M.Rec |
| | B3AR | P13945 | ADRB3 | 246 | Yes | 0.24 | 16 | 7 | M.Rec |
| | NaDST | P31645 | SLC6A4 | 228 | Yes | 0.23 | 367 | 45 | Trans |
| | CHR | P06276 | BCHE | 1914 | Yes | 0.23 | 74 | 6 | Enz. |
| | ACHR | P22303 | ACHE | 220 | No | 0.23 | 74 | 6 | Enz. |
| 013 | 5HTR1A | P08908 | HTR1A | 214 | No | 0.34 | 14 | 211 | M.Rec |
| | 5HTR1B | P28222 | HTR1B | 1898 | Yes | 0.34 | 15 | 217 | M.Rec |
| | 5HTR6 | P50406 | HTR6 | 3371 | No | 0.3 | 2 | 47 | M.Rec |
| | 5HTR2A | P28223 | HTR2A | 224 | Yes | 0.28 | 13 | 107 | M.Rec |
| | B2AR | P07550 | ADRB2 | 210 | No | 0.28 | 32 | 6 | M.Rec |
| | B1AR | P08588 | ADRB1 | 213 | Yes | 0.28 | 32 | 6 | M.Rec |
| | B3AR | P13945 | ADRB3 | 246 | No | 0.28 | 32 | 6 | M.Rec |
| | 5HTR1D | P28221 | HTR1D | 1983 | No | 0.28 | 6 | 153 | M.Rec |
| | 5HTR1E | P28566 | HTR1E | 2182 | Yes | 0.28 | 5 | 146 | M.Rec |
| | 5HTR1F | P30939 | HTR1F | 1805 | Yes | 0.28 | 5 | 146 | M.Rec |
| | FAAH1 | O00519 | FAAH | 2243 | No | 0.27 | 7 | 11 | Enz. |
| | NaDNT | P23975 | SLC6A2 | 222 | No | 0.21 | 29 | 24 | Trans |
| | NaDST | P31645 | SLC6A4 | 228 | No | 0.21 | 32 | 52 | Trans |
| | NaCIDGT1 | P48067 | SLC6A9 | 2337 | Yes | 0.21 | 39 | 24 | Trans |
| | NaDDT | Q01959 | SLC6A3 | 238 | No | 0.21 | 29 | 24 | Trans |
| 014 | 5HTR1A | P08908 | HTR1A | 214 | No | 0.56 | 389 | 203 | M.Rec |
| | 5HTR1D | P28221 | HTR1D | 1983 | No | 0.56 | 118 | 142 | M.Rec |
| | 5HTR1B | P28222 | HTR1B | 1898 | No | 0.56 | 408 | 209 | M.Rec |
| | 5HTR1E | P28566 | HTR1E | 2182 | Yes | 0.56 | 86 | 135 | M.Rec |
| | 5HTR1F | P30939 | HTR1F | 1805 | Yes | 0.56 | 86 | 135 | M.Rec |
| | 5HTR6 | P50406 | HTR6 | 3371 | No | 0.51 | 177 | 47 | M.Rec |
| | 5HTR2A | P28223 | HTR2A | 224 | Yes | 0.44 | 359 | 99 | M.Rec |
| | CHR | P06276 | BCHE | 1914 | Yes | 0.36 | 112 | 6 | Enz. |
| | ACHR | P22303 | ACHE | 220 | No | 0.36 | 112 | 6 | Enz. |
| | 5HTR2C | P28335 | HTR2C | 225 | Yes | 0.31 | 235 | 73 | M.Rec |
| | 5HTR2B | P41595 | HTR2B | 1833 | No | 0.31 | 235 | 73 | M.Rec |
| | NaDNT | P23975 | SLC6A2 | 222 | Yes | 0.31 | 727 | 26 | Trans |
| | NaDST | P31645 | SLC6A4 | 228 | No | 0.31 | 654 | 60 | Trans |
| | NaCIDGT1 | P48067 | SLC6A9 | 2337 | Yes | 0.31 | 402 | 26 | Trans |
| | NaDDT | Q01959 | SLC6A3 | 238 | No | 0.31 | 727 | 26 | Trans |
| 015 | 5HTR1A | P08908 | HTR1A | 214 | No | 0.33 | 296 | 109 | M.Rec |
| | 5HTR1B | P28222 | HTR1B | 1898 | No | 0.33 | 301 | 110 | M.Rec |
| | 5HTR1D | P28221 | HTR1D | 1983 | No | 0.31 | 91 | 62 | M.Rec |
| | 5HTR1E | P28566 | HTR1E | 2182 | Yes | 0.31 | 80 | 61 | M.Rec |
| | 5HTR1F | P30939 | HTR1F | 1805 | Yes | 0.31 | 80 | 61 | M.Rec |
| | MARM4 | P08173 | CHRM4 | 1821 | Yes | 0.29 | 235 | 2 | M.Rec |
| | MARM5 | P08912 | CHRM5 | 2035 | Yes | 0.29 | 195 | 2 | M.Rec |
| | MARM1 | P11229 | CHRM1 | 216 | No | 0.29 | 232 | 3 | M.Rec |
| | MARM3 | P20309 | CHRM3 | 245 | No | 0.29 | 224 | 2 | M.Rec |
| | NaDNT | P23975 | SLC6A2 | 222 | Yes | 0.27 | 776 | 22 | Trans |
| | NaDST | P31645 | SLC6A4 | 228 | No | 0.27 | 802 | 46 | Trans |
| | NaCIDGT1 | P48067 | SLC6A9 | 2337 | Yes | 0.27 | 554 | 21 | Trans |
| | NaDDT | Q01959 | SLC6A3 | 238 | Yes | 0.27 | 776 | 22 | Trans |
| | NaCIDGT2 | Q9Y345 | SLC6A5 | 3060 | Yes | 0.27 | 554 | 21 | Trans |
| | NaDPT | Q99884 | SLC6A7 | | Yes | 0.27 | 554 | 21 | Trans |
| 016 | 5HTR2A | P28223 | HTR2A | 224 | No | 0.41 | 569 | 80 | M.Rec |
| | 5HTR2C | P28335 | HTR2C | 225 | No | 0.41 | 345 | 54 | M.Rec |
| | 5HTR2B | P41595 | HTR2B | 1833 | No | 0.41 | 345 | 54 | M.Rec |
| | 5HTR6 | P50406 | HTR6 | 3371 | No | 0.41 | 220 | 29 | M.Rec |
| | 5HTR1A | P08908 | HTR1A | 214 | No | 0.39 | 829 | 140 | M.Rec |
| | 5HTR1D | P28221 | HTR1D | 1983 | No | 0.39 | 358 | 114 | M.Rec |
| | 5HTR1B | P28222 | HTR1B | 1898 | Yes | 0.39 | 878 | 146 | M.Rec |
| | 5HTR1E | P28566 | HTR1E | 2182 | Yes | 0.39 | 290 | 108 | M.Rec |
| | 5HTR1F | P30939 | HTR1F | 1805 | No | 0.39 | 290 | 108 | M.Rec |
| | CHR | P06276 | BCHE | 1914 | Yes | 0.29 | 302 | 4 | Enz. |
| | ACHR | P22303 | ACHE | 220 | No | 0.29 | 302 | 4 | Enz. |
| | MARM2 | P08172 | CHRM2 | 211 | Yes | 0.26 | 182 | 1 | M.Rec |
| | MARM4 | P08173 | CHRM4 | 1821 | No | 0.26 | 213 | 1 | M.Rec |
| | MARM3 | P20309 | CHRM3 | 245 | Yes | 0.26 | 167 | 1 | M.Rec |
| | MTOR | P35372 | OPRM1 | 233 | No | 0.26 | 774 | 36 | M.Rec |
| 017 | 5HTR1A | P08908 | HTR1A | 214 | No | 0.92 | 61 | 481 | M.Rec |
| | 5HTR1B | P28222 | HTR1B | 1898 | Yes | 0.92 | 63 | 488 | M.Rec |
| | 5HTR2A | P28223 | HTR2A | 224 | Yes | 0.92 | 93 | 220 | M.Rec |
| | 5HTR6 | P50406 | HTR6 | 3371 | No | 0.92 | 45 | 218 | M.Rec |
| | 5HTR1D | P28221 | HTR1D | 1983 | No | 0.86 | 38 | 391 | M.Rec |
| | 5HTR1E | P28566 | HTR1E | 2182 | Yes | 0.86 | 34 | 379 | M.Rec |
| | 5HTR1F | P30939 | HTR1F | 1805 | Yes | 0.86 | 34 | 379 | M.Rec |
| | 5HTR2C | P28335 | HTR2C | 225 | No | 0.82 | 71 | 167 | M.Rec |
| | 5HTR2B | P41595 | HTR2B | 1833 | No | 0.82 | 71 | 167 | M.Rec |
| | TDNAP1 | Q9NUW8 | TDP1 | 1075138 | No | 0.77 | 8 | 12 | Enz. |
| | MBLP1 | Q9NR56 | MBNL1 | 1293317 | No | 0.71 | 8 | 27 | Unc |
| | MBLP2 | Q5VZF2 | MBNL2 | | Yes | 0.71 | 8 | 27 | Unc |
| | MBLP3 | Q9NUK0 | MBNL3 | | Yes | 0.71 | 8 | 27 | Unc |
| | 5HTR7 | P34969 | HTR7 | 3155 | No | 0.68 | 36 | 41 | M.Rec |
| | D(2)DR | P14416 | DRD2 | 217 | Yes | 0.66 | 196 | 305 | M.Rec |
| 018 | 5HTR1A | P08908 | HTR1A | 214 | No | 0.84 | 61 | 629 | M.Rec |
| | 5HTR1B | P28222 | HTR1B | 1898 | Yes | 0.84 | 63 | 645 | M.Rec |
| | 5HTR2A | P28223 | HTR2A | 224 | Yes | 0.84 | 96 | 241 | M.Rec |
| | 5HTR6 | P50406 | HTR6 | 3371 | No | 0.84 | 46 | 246 | M.Rec |
| | 5HTR1D | P28221 | HTR1D | 1983 | No | 0.72 | 39 | 490 | M.Rec |
| | 5HTR1E | P28566 | HTR1E | 2182 | Yes | 0.72 | 35 | 469 | M.Rec |
| | 5HTR1F | P30939 | HTR1F | 1805 | Yes | 0.72 | 35 | 469 | M.Rec |
| | 5HTR2C | P28335 | HTR2C | 225 | No | 0.7 | 73 | 184 | M.Rec |
| | 5HTR2B | P41595 | HTR2B | 1833 | Yes | 0.7 | 73 | 184 | M.Rec |
| | D(2)DR | P14416 | DRD2 | 217 | No | 0.65 | 189 | 430 | M.Rec |
| | D(3)DR | P35462 | DRD3 | 234 | No | 0.65 | 136 | 217 | M.Rec |
| | NaDNT | P23975 | SLC6A2 | 222 | No | 0.63 | 256 | 229 | Trans |
| | NaDST | P31645 | SLC6A4 | 228 | No | 0.63 | 245 | 342 | Trans |
| | NaDDT | Q01959 | SLC6A3 | 238 | No | 0.63 | 256 | 229 | Trans |
| | NaCIDGT1 | P48067 | SLC6A9 | 2337 | Yes | 0.63 | 183 | 217 | Trans |
| 019 | CHR | P06276 | BCHE | 1914 | Yes | 0.4 | 44 | 20 | Enz. |
| | ACHR | P22303 | ACHE | 220 | No | 0.4 | 44 | 20 | Enz. |
| | MARM1 | P11229 | CHRM1 | 216 | Yes | 0.4 | 42 | 46 | M.Rec |
| | MBLP1 | Q9NR56 | MBNL1 | 1293317 | No | 0.35 | 292 | 51 | Unc |
| | MBLP2 | Q5VZF2 | MBNL2 | | Yes | 0.35 | 292 | 51 | Unc |
| | MBLP3 | Q9NUK0 | MBNL3 | | Yes | 0.35 | 292 | 51 | Unc |
| | TPKJAK2 | O60674 | JAK2 | 2971 | No | 0.33 | 45 | 81 | TyrK |
| | TPKJAK1 | P23458 | JAK1 | 2835 | No | 0.33 | 45 | 81 | TyrK |
| | Non- | P29597 | TYK2 | 3553 | Yes | 0.33 | 45 | 81 | TyrK |
| | receptor | | | | | | | | |
| | tyrosine- | | | | | | | | |
| | protein | | | | | | | | |
| | kinase TYK2 | | | | | | | | |
| | TPKJAK3 | P52333 | JAK3 | 2148 | Yes | 0.33 | 45 | 81 | TyrK |
| | CP45011B1 | Mitchond | P15538 | CYP11B1 | CHEMBL1908 | No | 0.31 | 62 | Enz. |
| | CP45011B2 | Mitchond | P19099 | CYP11B2 | CHEMBL2722 | No | 0.31 | 62 | Enz. |
| | Cholesterol | Mitchond | P05108 | CYP11A1 | CHEMBL2033 | Yes | 0.31 | 62 | Enz. |
| | side-chain | | | | | | | | |
| | cleavage | | | | | | | | |
| | enzyme | | | | | | | | |
| | Thromboxane- | P24557 | TBXAS1 | 1835 | No | 0.31 | 24 | 41 | Enz. |
| | A synthase | | | | | | | | |
| | MARM2 | P08172 | CHRM2 | 211 | Yes | 0.28 | 25 | 63 | M.Rec |
| 020 | 5HTR2A | P28223 | HTR2A | 224 | No | 1 | 76 | 193 | M.Rec |
| | 5HTR2C | P28335 | HTR2C | 225 | No | 1 | 71 | 151 | M.Rec |
| | 5HTR2B | P41595 | HTR2B | 1833 | No | 1 | 71 | 151 | M.Rec |
| | 5HTR1A | P08908 | HTR1A | 214 | Yes | 0.74 | 12 | 528 | M.Rec |
| | 5HTR1D | P28221 | HTR1D | 1983 | No | 0.74 | 17 | 444 | M.Rec |
| | 5HTR1B | P28222 | HTR1B | 1898 | No | 0.74 | 19 | 542 | M.Rec |
| | 5HTR1E | P28566 | HTR1E | 2182 | Yes | 0.74 | 10 | 425 | M.Rec |
| | 5HTR1F | P30939 | HTR1F | 1805 | Yes | 0.74 | 10 | 425 | M.Rec |
| | 5HTR6 | P50406 | HTR6 | 3371 | No | 0.74 | 6 | 184 | M.Rec |
| | MBLP#1 | Q9NR56 | MBNL1 | 1293317 | No | 0.74 | 1 | 19 | Unc |
| | MBLP#2 | Q5VZF2 | MBNL2 | | Yes | 0.74 | 1 | 19 | Unc |
| | MBLP#3 | Q9NUK0 | MBNL3 | | Yes | 0.74 | 1 | 19 | Unc |
| | Na-Dep | P23975 | SLC6A2 | 222 | Yes | 0.64 | 18 | 194 | Trans. |
| | noradrenaline | | | | | | | | |
| | Trans. | | | | | | | | |
| | Na-Dep | P31645 | SLC6A4 | 228 | No | 0.64 | 35 | 262 | Trans. |
| | serotonin | | | | | | | | |
| | Trans. | | | | | | | | |
| | Na-Dep | Q01959 | SLC6A3 | 238 | No | 0.64 | 18 | 194 | Trans. |
| | dopamine | | | | | | | | |
| | Trans. | | | | | | | | |

### Synthetic Routes

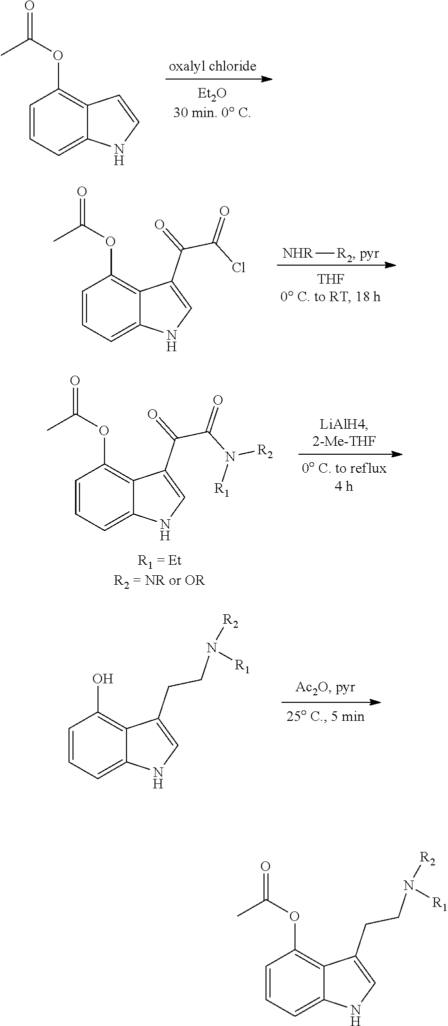

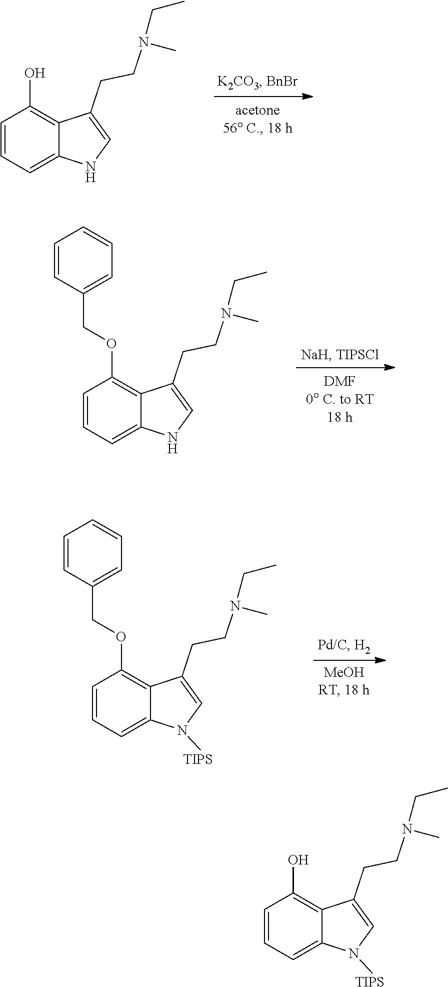

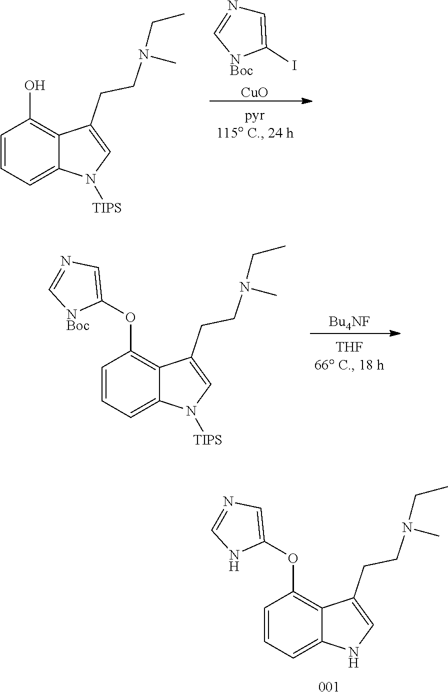

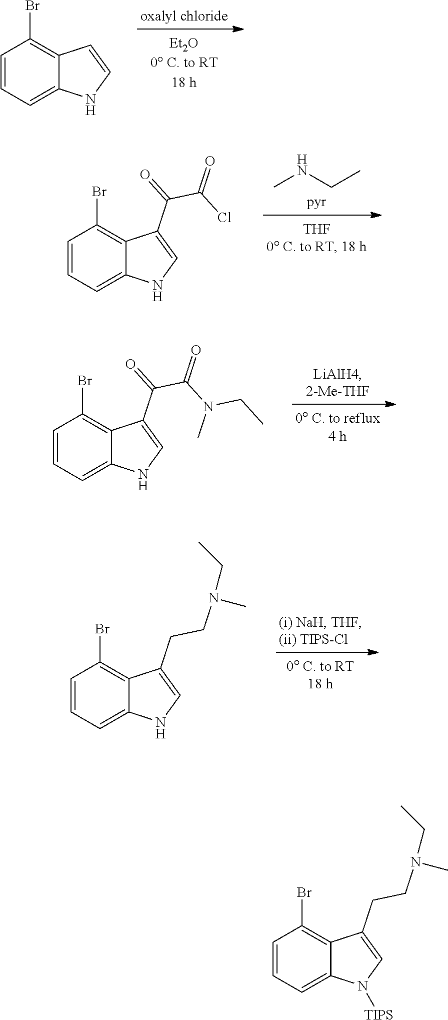

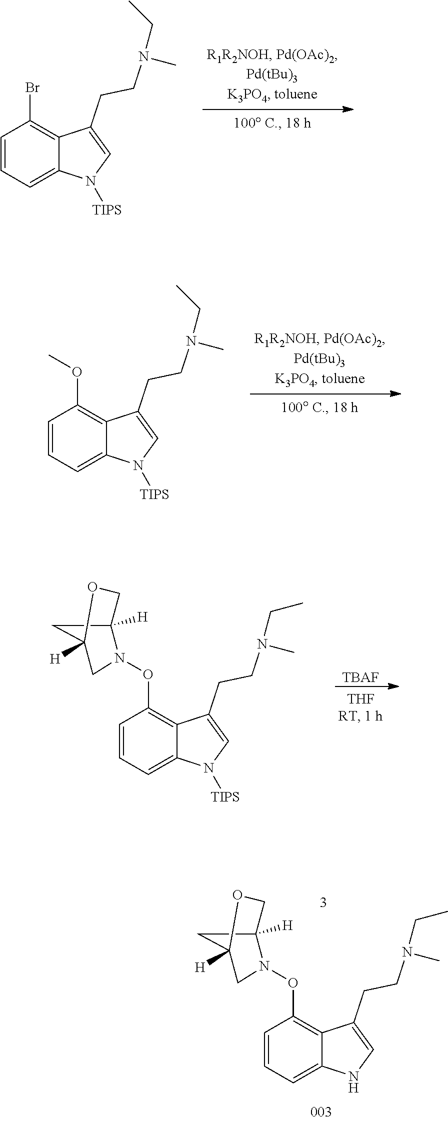

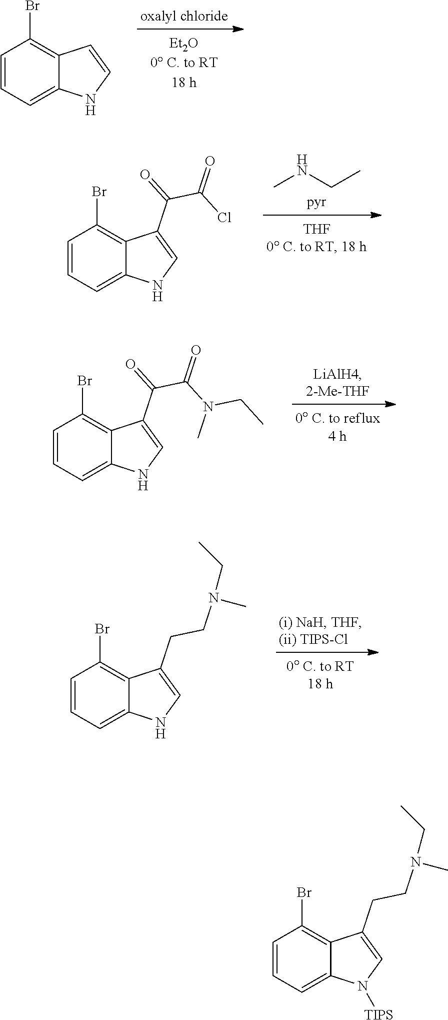

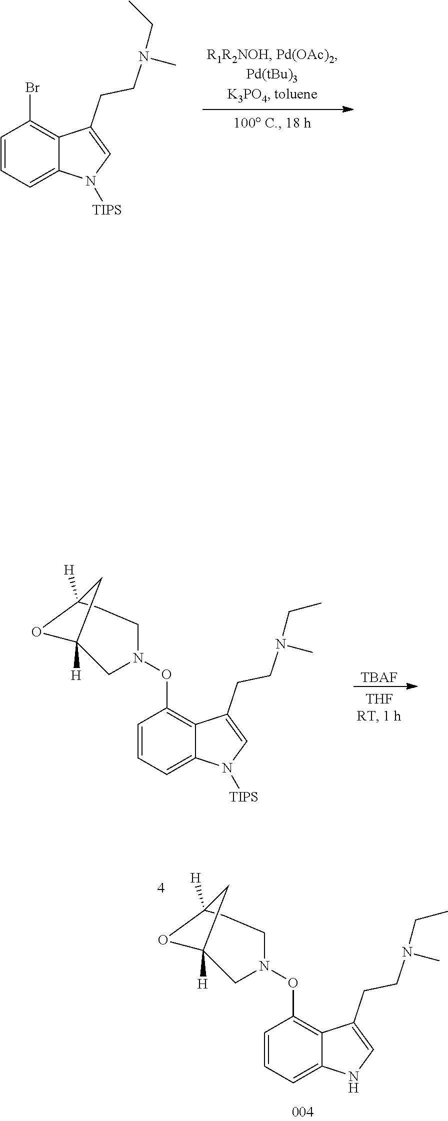

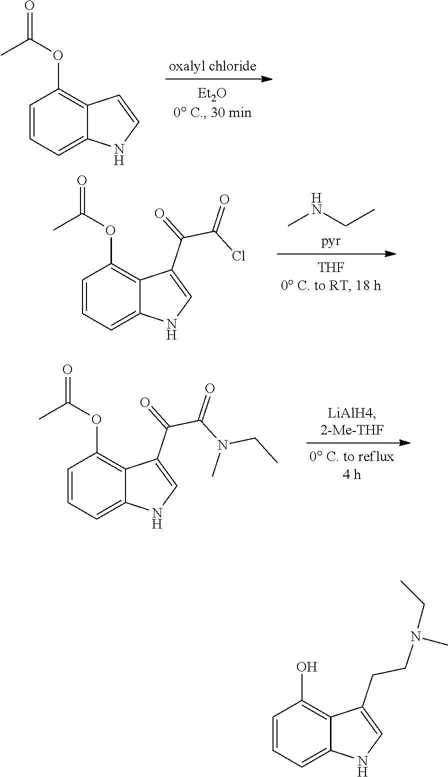

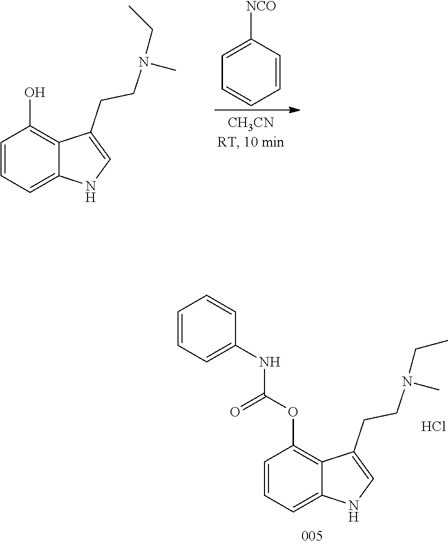

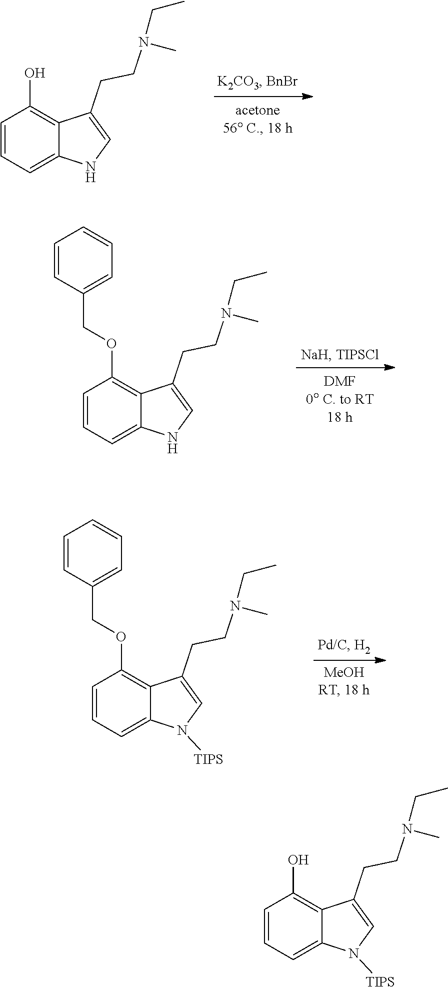

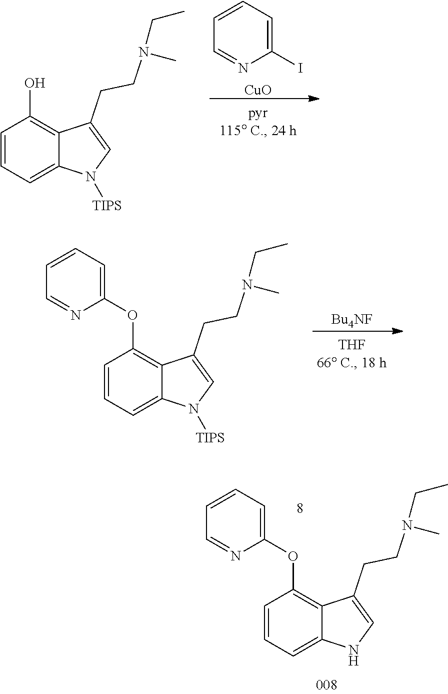

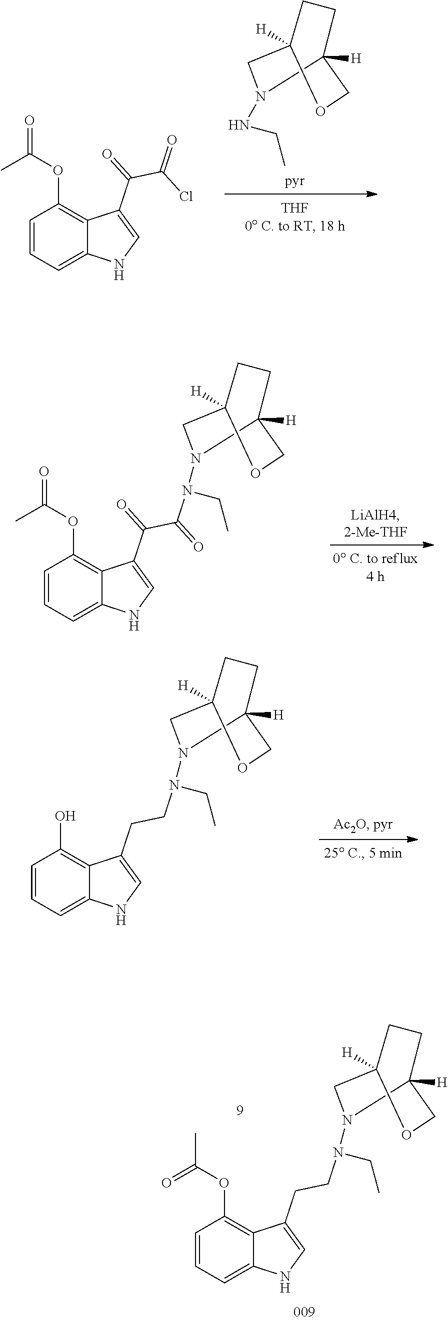

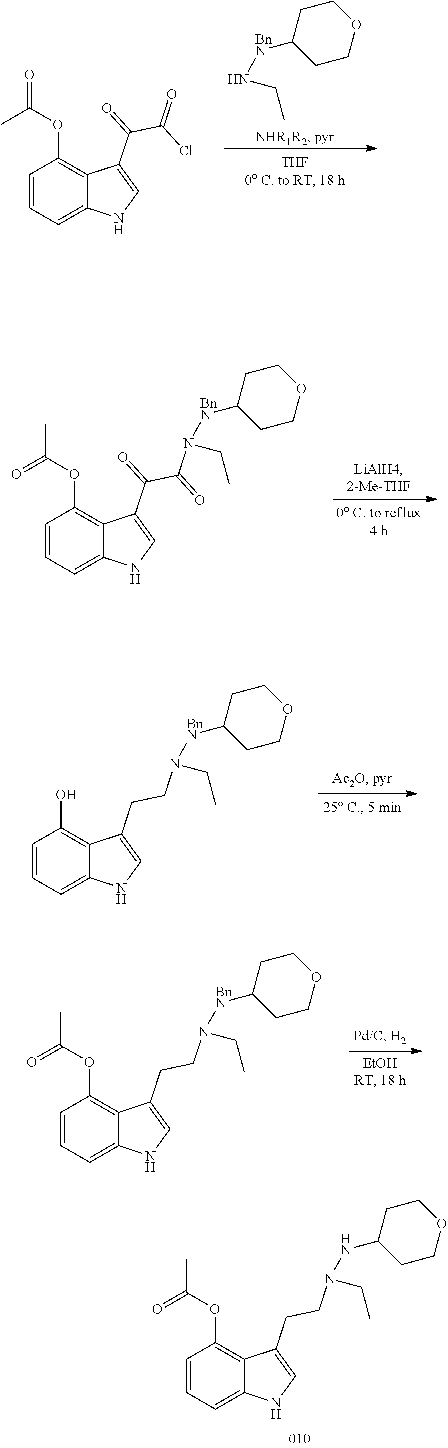

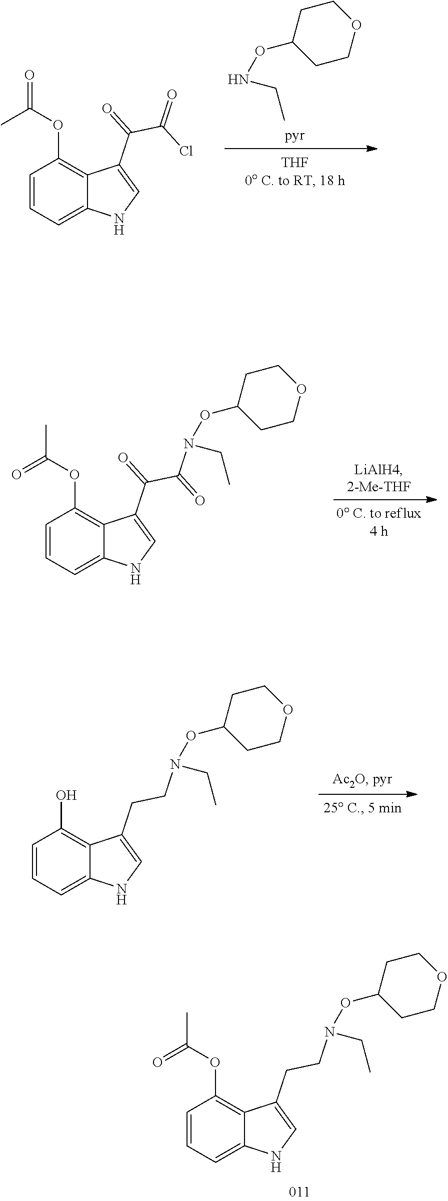

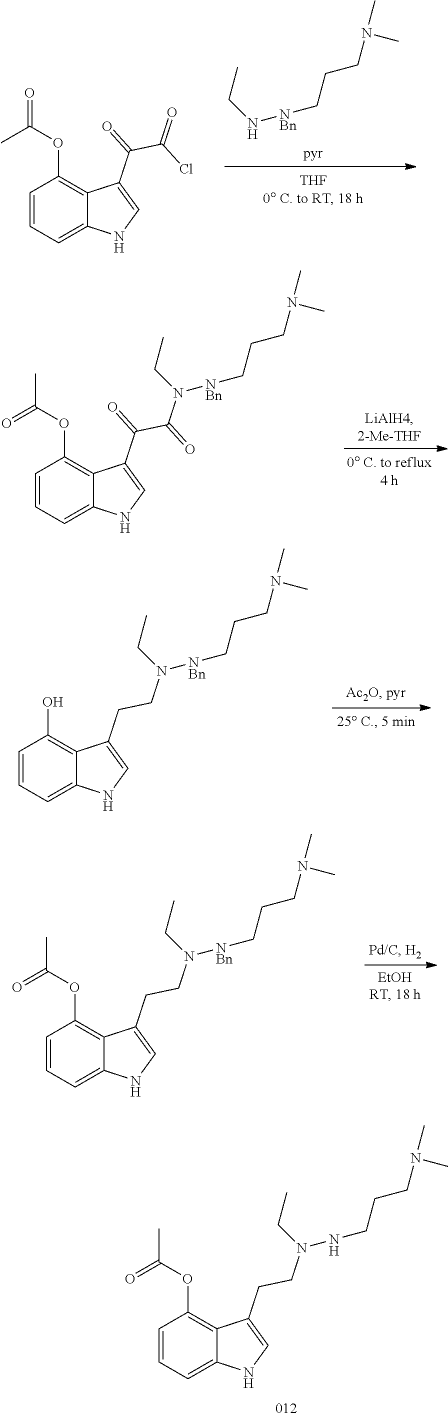

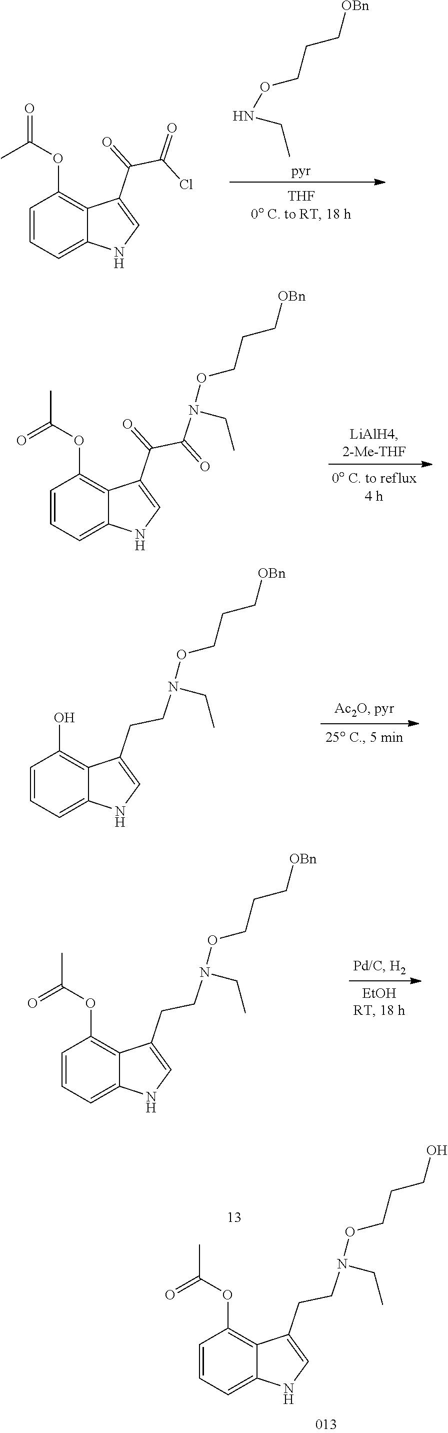

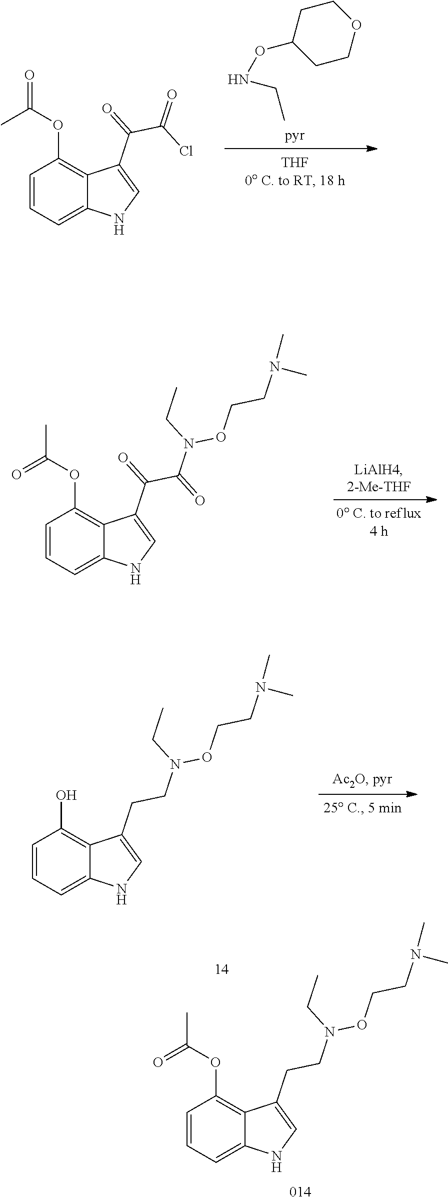

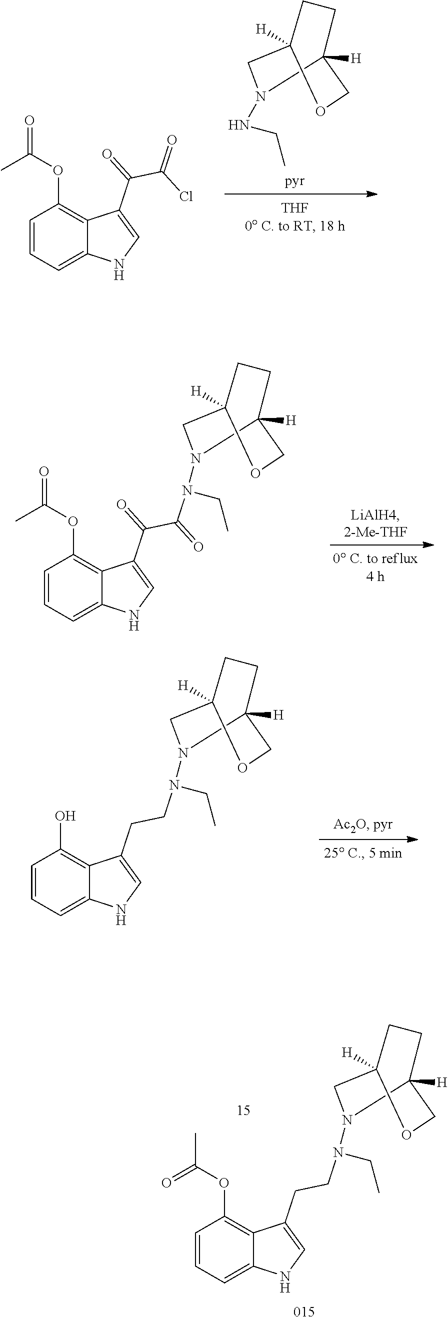

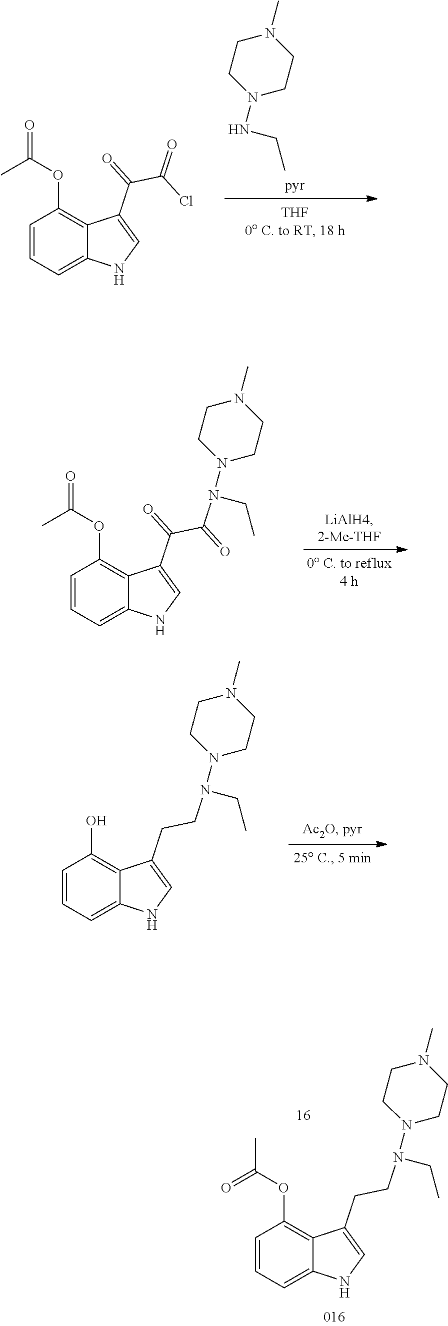

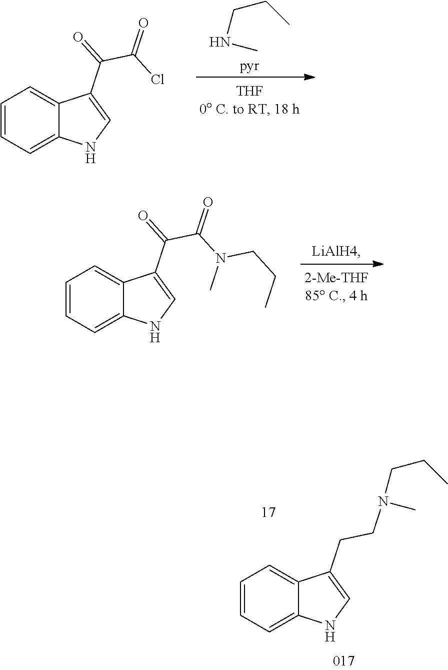

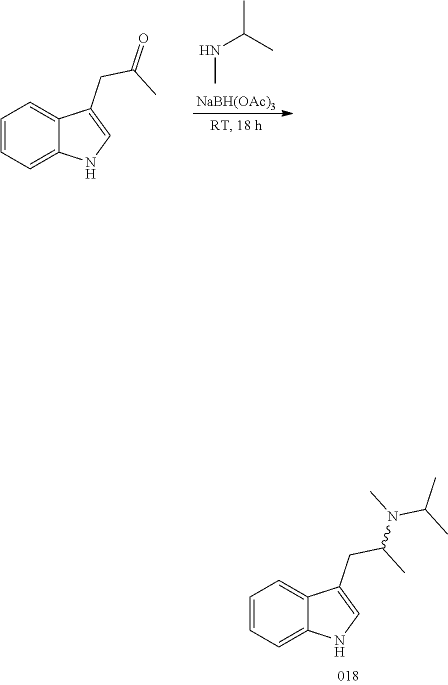

Salt formation is undertaken thereafter in the above routes as necessary, e.g. to give the benzoate, fumarate, citrate, acetate, succinate, halide, fluoride, chloride, bromide, iodide, oxalate, or triflate salt. For example, the addition of hydrogen chloride would provide the chloride salt and benzoic acid would give the benzoate salt.

In an embodiment, there is provided a method of synthesis of any of the herein described compounds. In an embodiment, there is provided a method of synthesis of compound 001, 002, 003, 004, 005, 006, 007, 008, 009, 010, 011, 012, 013, 014, 015, 016, 017, 018, 019 or 020 as herein disclosed.

### Detailed Synthesis of Compound 005

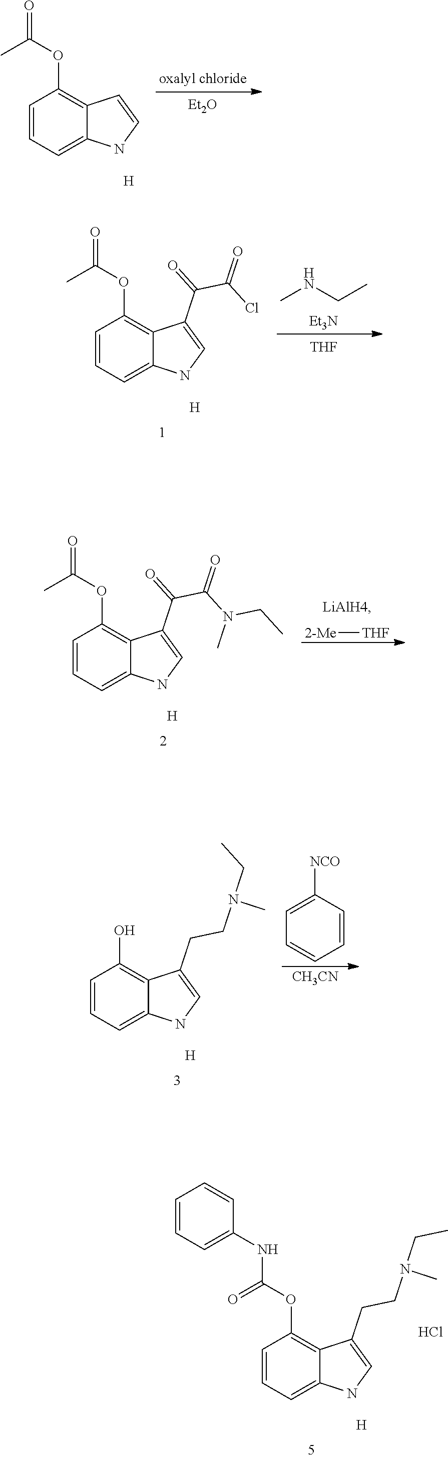

### 3-[2-(N-Methyl-N-ethylamino)-2-oxoethanoyl]-1H-indol-4-yl ethanoate (2

To a stirred suspension of 4-indolyl acetate (2.0 g, 11.4 mmol) in diethyl ether (11.4 mL) at 0° C. was added dropwise oxalyl chloride (2.2 g, 1.5 mL, 17.1 mmol), maintaining a reaction temperature under 5° C., and stirring was continued at 0° C. for 30 min.

The reaction mixture was diluted with hexane (23 mL) and cooled to −20° C. for 18 h. The yellow precipitate was collected by suction filtration, washed with hexane (3×5 mL) and dried to afford 1 as a yellow powder (2.73 g, 90%) that was used directly.

To a cooled suspension of 1 (2.73 g, 10.3 mmol) in THF (11 mL) at 0° C. was added dropwise N-ethylmethylamine (736 mg, 1.07 mL, 12.4 mmol) and pyridine (978 mg, 1.0 mL, 12.4 mmol) and the reaction mixture was allowed to warm to room temperature with stirring for 18 h.

The reaction mixture was concentrated and partitioned between water (25 mL) and ethyl acetate (25 mL). Layers were separated and the aqueous phase was extracted with ethyl acetate (2×25 mL). Combined organic layers were dried (Na₂SO₄) and concentrated to afford the title compound as a yellow powder (2.3 g, 77%) that was used without purification.

LC-MS (Method 2.5 basic): Rt 0.98 mins; MS m/z 287.3=[M−H]—(98% @254 nm)

### 3-[2-(N-Methyl-N-ethylamino)-2-oxoethanoyl]-1H-indol-4-yl ethanoate (3

To a stirred, cooled solution of 2 (2.3 g, 8.0 mmol) in 2-methyltetrahydrofuran (54 mL) at 0° C. was added dropwise lithium aluminium hydride solution (10.0 mL, 23.9 mmol, 2.4M in THF) over 10 min and the reaction mixture was heated to reflux for 3 h.

The mixture was cooled to 0° C. and quenched by dropwise addition of THF/water (1:4, 2.8 mL). Sodium sulfate (5.43 g), silica (2.73 g) and dichloromethane (24 mL) were added. The product was filtered, eluted with 10% methanol in dichloromethane (3×70 mL) and the filtrate was concentrated to afford a dark brown solid (1.4 g).

Purification by reversed-phase chromatography (C18, 32 g, 2 35 50% acetonitrile in aq. 0.1% ammonia solution) gave the title compound as a dark green/blue solid (802 mg, 46%).

LC-MS (Method 2.5 basic): Rt 1.01 min; MS m/z 287.3=[M+H]⁺ (100% @254 nm)

¹H NMR (500 MHz, DMSO-d₆) δ 11.10-11.05 (m, 1H), 10.58 (s, 1H), 6.92 (d, J=2.2 Hz, 2H), 6.80 (t, J=7.8 Hz, 1H), 6.74 (d, J=7.9 Hz, 1H), 6.26 (d, J=7.3 Hz, 1H), 2.88 (t, J=6.5 Hz, 2H), 2.61 (t, J=6.5 Hz, 2H), 2.45 (q, J=7.2 Hz, 2H), 2.24 (s, 3H), 0.98 (t, J=7.1 Hz, 3H).

### Phenylamino 3-12-(N-methyl-N-ethylamino)ethyl-1H-indole-4-carboxylate hydrochloride (005

To a stirred mixture of 3 (50 mg, 0.23 mmol) in acetonitrile was added phenyl isocyanate (27 mg, 25 μL, 0.23 mmol) and stirring was continued at room temperature for 10 min. The material was purified by reversed-phase chromatography by direct injection (C18 20 g, 15 45% acetonitrile in aq. 0.01% HCl) to afford the title compound as an off-white solid (42 mg, 54%).

LC-MS (Method 8 min acid): Rt 2.64 min; MS m/z 338.1=[M+H]⁺ (100% @254 nm)

¹H NMR (500 MHz, DMSO-d₆) δ 11.26 (br s, 1H), 10.43 (br s, 1H), 10.16 (br s, 1H), 7.59 (d, J=8.0 Hz, 2H), 7.37-7.31 (m, 2H), 7.28 (d, J=8.1 Hz, 2H), 7.13-7.03 (m, 2H), 6.84 (d, J=7.6 Hz, 1H), 3.24-3.11 (m, 3H), 3.08-2.99 (m, 1H), 2.75-2.70 (m, 3H), 1.15 (t, J=7.3 Hz, 3H).

In one embodiment there is provided a method of synthesis of compound 005.

In one embodiment there is provided a method of synthesis of Compound 005 as described above.

### Further Characterisation of Compounds 005, 008 and 018

cAMP

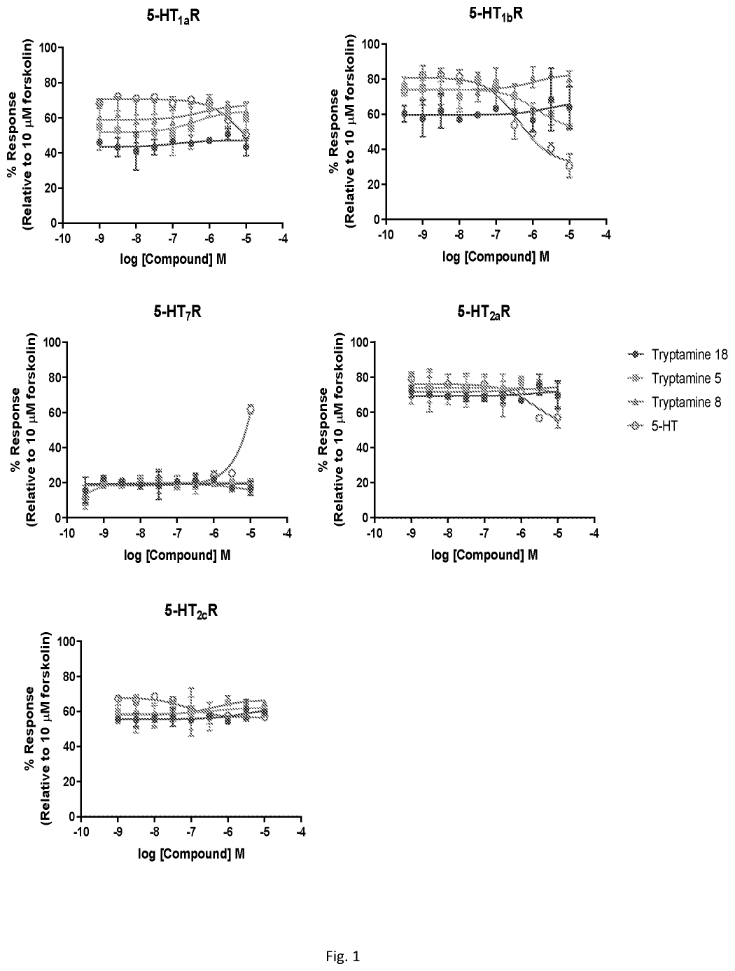

Compounds 005, 008 and 018 were assayed using a serotonin (1a, 1b, 2a, 2c and 7 receptors) cAMP assay, provided by Multispan. The reference used was 10 μM forskolin (a cAMP activator) to calculate the percentage relative response, serotonin was used as the control. The results can be seen in FIG. 1. The results indicate 005 may have some activation of the cAMP pathway and subsequent cascade.

### IP₁

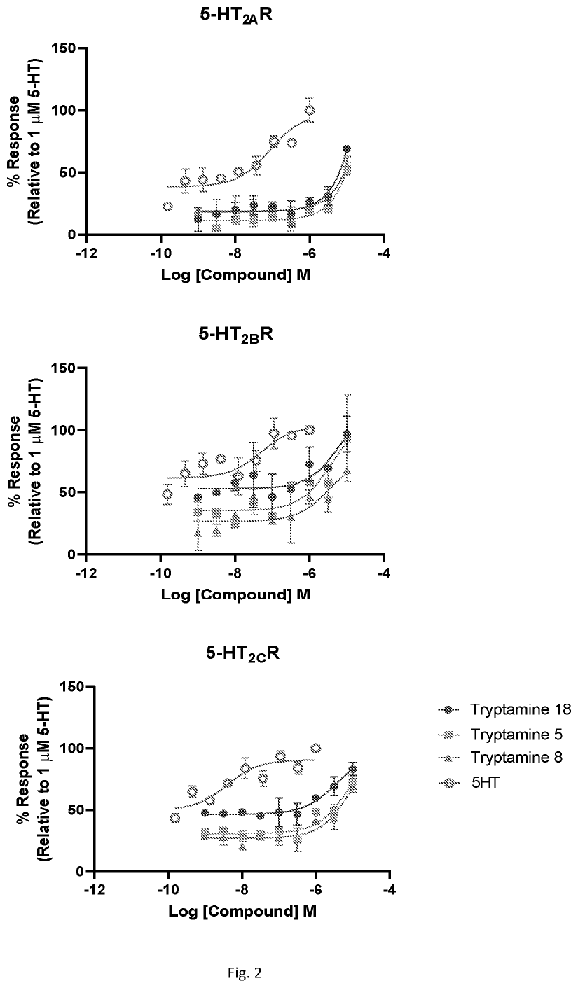

Compounds 005, 008 and 018 were assayed using a serotonin (2a, 2b and 2c receptors) inositol phosphate 1 (IP₁) assay, provided by Multispan. The reference used was 1 μM serotonin to calculate the percentage relative response. The results can be seen in FIG. 2. All compounds produced incomplete response curves in this assay, showing agonistic activity, implying that the compounds may not active any Gα_{q/11} pathway.

### Ca²⁺

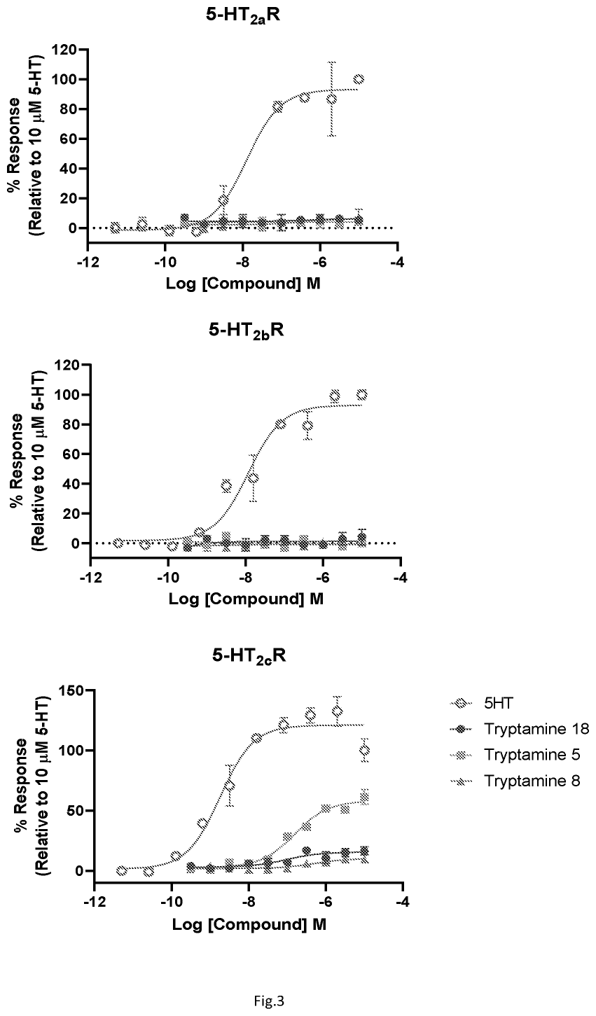

Compounds 005, 008 and 018 were assayed using a serotonin (2a, 2b and 2c receptors) calcium (Ca²⁺) assay, provided by Multispan. The reference used was 10 μM serotonin to calculate the percentage relative response in relative light units (RLU). The results can be seen in FIG. 3. Some activity for compound 005 in the 2c receptor assay was detected.

### B-Arrestin

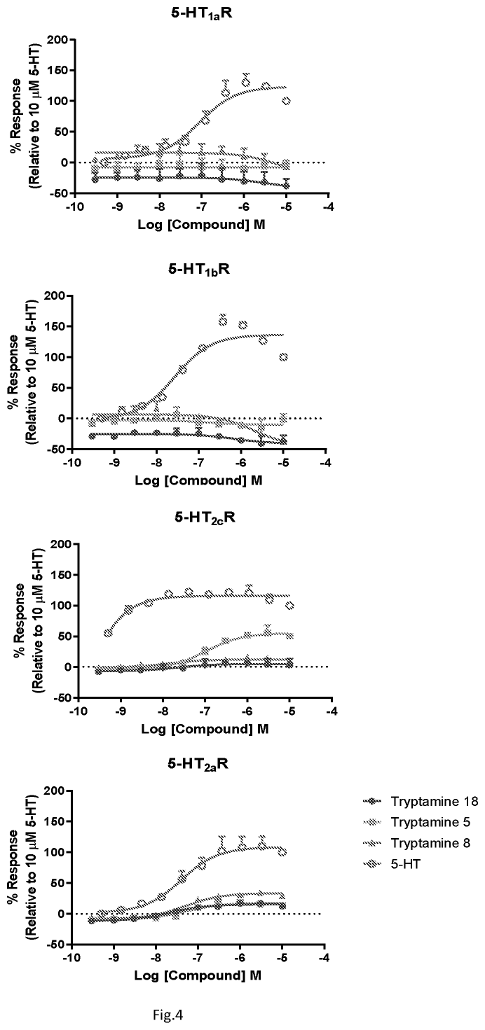

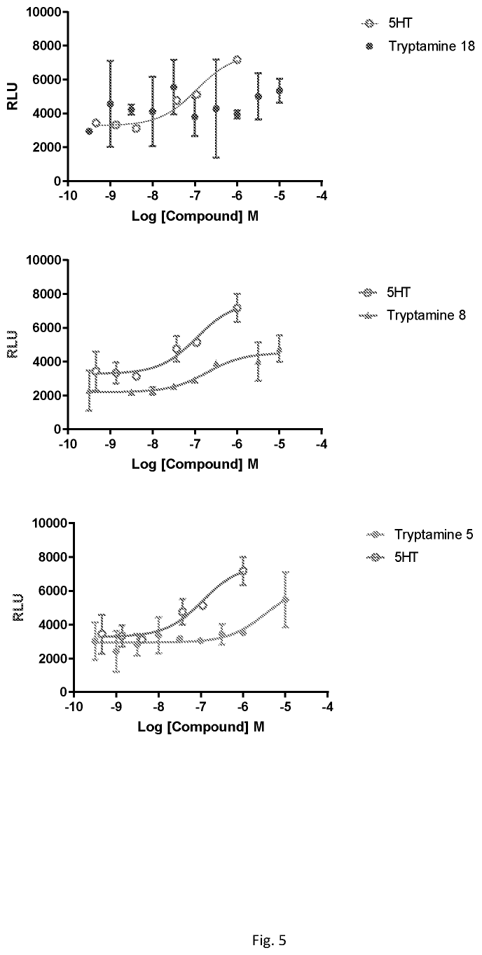

Compounds 005, 008 and 018 were assayed using a serotonin (1a, 1b, 2a and 2c receptors) B-arrestin assay, provided by DiscoverX. The reference used was 10 μM serotonin to calculate the percentage relative response. The results can be seen in FIG. 4. Some activity for all compounds in the 2a receptor activity was detected, whilst only compound 005 appeared to have activity in the 2c receptor assay. The compounds were also assayed in a serotonin 2b receptor B-arrestin assay, the results of which can be seen in FIG. 5.

### Reagent Information

### B-Arrestin:

HTR1A PathHunter® eXpress HTR1A CHO-K1 β-Arrestin GPCR Assay 93-0696E2CP0M 200 dp (2×96-well)
HTR1B PathHunter® eXpress HTR1B U2OS β-Arrestin GPCR Assay 93-0697E3CP6M 200 dp (2×96-well)
HTR2A PathHunter® eXpress HTR2A U2OS β-Arrestin GPCR Assay 93-0401E3CP19M 200 dp (2×96-well)
HTR2C PathHunter® eXpress HTR2C U2OS β-Arrestin GPCR Assay 93-0289E3CP3M 200 dp (2×96-well)
Multispan β-arrestin assay with CHO-K1-5HT_{2b}R cells—Catalog C1350-1a

### Homogeneous Time Resolved Fluorescence (HTRF) and Calcium:

IP-One Gq kit—cisbio—cat. No. 62IPAPEB
cAMP Gs dynamic kit—cisbio—cat. No. 62AM4PEC
FLIPR calcium 6 assay explorer kit—VWR—cat. No. MLDVR8190

### Cells Used in HTRF and Calcium Assays:

MULTISCREEN™ HEK293T Cell Line Stably Expressing Human 5-HT1A Receptor, Catalog DC1319a
MULTISCREEN™ HEK293T Cell Line Stably Expressing Human 5-HT1B Receptor, Catalog DC1320a
MULTISCREEN™ HEK293T Cell Line Stably Expressing Human 5HT1B Receptor, Catalog DC1320a
MULTISCREEN™ CHO-K1 Cell Line Stably Expressing Human 5-HT2A Receptor, Catalog DC1324-1
MULTISCREEN™ CHO-K1 Cell Line Stably Expressing Human 5-HT2B Receptor, Catalog DC1325-1
MULTISCREEN™ CHO-K1 Cell Line Stably Expressing Human 5-HT2C Receptor, Catalog DC1326-1
MULTISCREEN™ HEK293T Cell Line Stably Expressing Human 5-HT7 Receptor, Catalog DC1334

For the B-arrestin assays the cells came with the kits apart from the 2b receptor assay (Valiscreen serotonin 5HT-2B (human) cell line—ES-314-C, Perkin Elmer)

1. A compound of Formula (1) wherein: 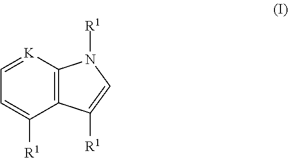
R¹ is selected from H or C₁₋₆ alkyl; and
R² is CH₂—CHR⁴—NR⁵R⁶, wherein
R⁴ is selected from H or C₁₋₆ alkyl, wherein
R⁵ is selected from H or C₁₋₆ alkyl, wherein
R⁶ is:
d) 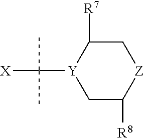
wherein X is a bond, Y is N, Z is O and R⁷ and R⁸ together from a bringing group CH₂CH₂; or
e) 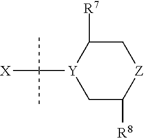
wherein X is NH or O, Y is CH, Z is O and R⁷ and R⁸ are H; or
f) NH(CH₂)₃N(CH₃)₂, O(CH₂)OH or O(CH₂)₂N(CH₃)₂.
R³ is selected from H or L—R⁹, wherein
L is selected from O, N, NH, O—O, CH₂, CH₂CH₂, O—O—CH₂CH₂, CO, OCO, COO or OCONH,
wherein
R⁹ is selected from H, C₁₋₆ alkyl, COOH, OH, COO—, O—, C₆-C₁₀ aromatic, C₃-C₁₀ heteroaromatic or heterocyclic group comprising one, two or three heteroatoms independently selected from O and N; and
K is CH;
or is a pharmaceutically acceptable salt thereof.

2. (canceled)

3. A compound of claim 1, wherein R¹ is H, R² is CH₂—CHR⁴—NR⁵R⁶, R³ is H or L—R⁹.

4. A compound of claim 1, wherein R⁴ is H or methyl.

5. A compound of claim 1, wherein R⁵ is ethyl or isopropyl.

6. A compound of claim 1, wherein R⁴ is H and R⁵ is ethyl.

7. A compound of claim 1, wherein R³ is L—R⁹ and wherein L is O, O—O—CH₂CH₂, OCO or OCONH.

8. A compound of claim 1, wherein R³ is L—R⁹ and wherein R⁹ is OH, COCH₃, imidazole, pyrrolidine, piperidine, analine, pyridine, morpholine or 6-oxa-3-azabicyclo[3.1.1]heptane.

9. A compound of claim 1, wherein R³ is OCOCH₃.

10-22. (canceled)

23. A pharmaceutical composition comprising a pharmaceutically effective amount of a compound as claimed in claim 1 and one or more pharmaceutically acceptable carriers or excipients.

24. The pharmaceutical composition of claim 23 for use in a method of treatment of a condition in a subject in need thereof, wherein the condition is selected from conditions caused by dysfunctions of the central nervous system, conditions caused by dysfunctions of the peripheral nervous system, conditions benefiting from sleep regulation, conditions benefiting from analgesics, migraines, trigeminal autonomic cephalgias, conditions benefiting from neurogenesis, conditions benefiting from anti-inflammatory treatment, depression, anxiety, substance use disorder, addictive disorder, gambling disorder, eating disorders, obsessive-compulsive disorders, or body dysmorphic disorders, optionally the condition is SUNCT and/or SUNA.

25. The pharmaceutical composition of claim 24, wherein the method of treatment is a method of treatment of depression.
